(19) United States
(12) Patent Application Publication
(10) Pub. No.: US 2026/0229351 A1
(43) Pub. Date: Aug. 6, 2026

(54) Integrated AI-driven System for Early Detection of Cervical and Breast Cancer Using Multi-Modal Imaging, Liquid Biopsy, and Epigenetic Biomarkers.

(71) Applicant: FURONG BEI, San Gabriel, CA (US)

(72) Inventor: FURONG BEI, San Gabriel, CA (US)

(21) Appl. No.: 19/067,907

(22) Filed: Mar. 2, 2025

(51) Int. Cl. G16H 50/20; G16H 30/20

(52) U.S. Cl. CPC G16H 50/20; G16H 30/20

(57) ABSTRACT

A quantum-dot-enhanced CRISPR-Cas13a microfluidic molecular triage and clinical routing system for rapid high-risk human papillomavirus genotyping in cervical and breast cancer care pathways includes a disposable microfluidic cartridge and a reader for rapid genotyping of high-risk human papillomavirus from cervical samples. The cartridge includes a sample inlet, lysis zone, CRISPR reaction zone, optical detection zone, and waste reservoir. At least one reaction microchannel has a width of about 100 micrometers and a depth of about 50 micrometers. A CRISPR-Cas13a assay is incubated at about 37 degrees Celsius for about 15 minutes. Fluorescence from a quantum-dot reporter having a peak emission at about 605 nanometers is detected to generate a same-visit triage output indicating HPV16/18 positive, other high-risk HPV positive, negative, invalid, or repeat-test recommended. Optional communications features support secure transmission of results or run metadata to patient, provider, emergency, or reimbursement interfaces.

### CROSS-REFERENCE TO RELATED APPLICATIONS

This application is organized as a concentrated, product-centered filing derived from subject matter previously associated with application Ser. No. 19/067,907 filed Mar. 2, 2025, while re-presenting the invention as a coherent cartridge-and-reader platform directed to rapid high-risk HPV genotyping, same-visit cervical triage, and optional networked clinical routing.

The disclosure is intentionally structured to improve written-description support, claim clarity, manufacturing relevance, licensing readiness, and transaction value while preserving support for continuation, divisional, continuation-in-part, foreign counterpart, and portfolio-packaging strategies to the extent permitted by applicable law and supported by the issued or pending prosecution record.

The presently claimed core invention remains centered on a concrete cartridge-reader-kit architecture and a same-visit cervical molecular triage workflow, while the broader specification preserves optional deployment embodiments involving secure routing, specialist review, patient and provider portals, breast-health follow-up, liquid-biopsy linkage, and epigenetic workflow support without requiring such optional embodiments to define the narrowest claim core.

Related applications, if later identified in an Application Data Sheet (ADS), may provide family, background, or complementary disclosure context; however, only those applications expressly identified in the ADS are relied upon for any domestic benefit or priority claim.

### STATEMENT REGARDING FEDERALLY SPONSORED RESEARCH OR DEVELOPMENT

Not applicable.

### REFERENCE TO A SEQUENCE LISTING, A TABLE, OR A COMPUTER PROGRAM LISTING APPENDIX

Not applicable.

### FIELD OF THE INVENTION

The invention relates generally to molecular triage systems for rapid high-risk HPV genotyping in cervical and breast cancer care pathways, including microfluidic sample-to-answer systems, fluorescence-based detection, cartridge-and-reader architectures, and clinical routing workflows configured for same-visit decision support.

More particularly, the invention relates to a portable or clinic-deployable platform for rapid genotyping of high-risk human papillomavirus from cervical samples, where a disposable microfluidic cartridge cooperates with a temperature-controlled reader, optical detection components, processor-executed quality-control logic, and optional communications features to provide a molecular triage output that can enter cervical and breast cancer care pathways.

In especially useful embodiments, the invention is embodied as a compact commercial module that can be built, validated, transferred, licensed, manufactured, and deployed in cancer-screening and triage programs while also supporting optional network-enabled routing to patient-facing, provider-facing, emergency-coordination, or reimbursement interfaces and optional integration with adjunct cervical and breast cancer care workflows.

### BACKGROUND OF THE INVENTION

Persistent infection by high-risk human papillomavirus is associated with cervical precancer and cervical cancer. Screening programs frequently seek accurate identification of HPV16, HPV18, and other high-risk HPV classes because such information influences follow-up scheduling, referral pathways, repeat testing intervals, and patient counseling.

Central-laboratory nucleic-acid testing systems can provide clinically meaningful sensitivity and specificity, but such systems often depend on expensive instruments, trained technical staff, controlled laboratory logistics, batch-based workflows, and delays between sample collection and actionable results. These characteristics can be incompatible with outpatient clinics, mobile screening sites, low-resource settings, retail health sites, or dispersed public-health campaigns.

In many real-world care pathways, specimen collection, transport to a remote laboratory, accessioning, batched processing, report routing, and delayed patient visibility can extend the interval between collection and actionable review even when the analytical chemistry itself is capable of faster turnaround. This workflow friction reinforces the need for a compact sample-to-answer architecture that can generate a same-visit molecular triage output closer to the point of collection.

A further challenge is that many rapid testing proposals oversimplify workflow at the expense of subtype discrimination, contamination control, robust internal controls, or integration of a practical optical reader. Many broader proposals also attempt to combine too many independent clinical ambitions into a single undifferentiated platform narrative, which can weaken clarity, enablement, prosecution posture, and transaction usability.

There remains a need for a cartridge architecture that uses small reagent volumes, supports closed waste handling, is compatible with cervical sample inputs, allows reproducible temperature control, and presents stable optical geometry for compact fluorescence measurement.

There also remains a need for an architecture that does not stop at generating a molecular result but instead can securely route the result, under appropriate policy and privacy controls, to patient-facing portals, provider-facing portals, specialist-review workflows, emergency-coordination endpoints, pricing interfaces, or reimbursement-support interfaces when such routing is commercially or clinically desirable.

There further remains a need for an implementable point-of-care women's health molecular platform that is narrow enough to be examined as a real device invention, yet rich enough to support follow-on claiming directed to cartridge configurations, reader assemblies, control logic, kit formats, consumables, commercialization variants, and optional adjunct pathways involving AI-assisted cytology, breast-health follow-up, liquid biopsy, or epigenetic biomarkers.

### SUMMARY OF THE INVENTION

In one aspect, the invention provides a quantum-dot-enhanced CRISPR-Cas13a microfluidic molecular triage and clinical routing system for rapid high-risk HPV genotyping in cervical and breast cancer care pathways, including a disposable microfluidic cartridge and a reader. The cartridge includes a sample inlet, lysis zone, reaction zone, optical detection zone, and waste reservoir. The reader includes a cartridge-receiving bay, temperature-control components, an excitation-and-detection optical assembly, and a processor configured to generate a triage output from measured fluorescence.

In useful embodiments, at least one reaction microchannel has a width of about 100 micrometers and a depth of about 50 micrometers, a CRISPR-Cas13a assay is incubated at about 37 degrees Celsius for about 15 minutes, and fluorescence is detected from a quantum-dot reporter having an emission peak at about 605 nanometers.

In preferred embodiments, the processor generates one of the following results: HPV16/18 positive, other high-risk HPV positive, negative, invalid, or repeat-test recommended. The system may suppress substantive result issuance when an internal control fails a predetermined acceptance criterion.

In another aspect, the invention provides a method in which a cervical sample is introduced into the cartridge, lysed on-chip or in a coupled chamber, transferred into one or more reaction channels, incubated under temperature control, optically interrogated, checked against control criteria, and converted into a same-visit triage result.

In another aspect, the invention provides a kit including disposable cartridges, reagents, optional controls, cervical collection components, and instructions for use with a portable or benchtop reader.

In selected embodiments, the system additionally supports optional network-enabled deployment, including patient-facing, provider-facing, emergency-coordination, pricing, and reimbursement interfaces, without requiring such interfaces to define the narrowest technical novelty of the presently claimed hardware and workflow.

In selected embodiments, the system interoperates with optional adjunct inputs including AI-assisted cervical cytology review, breast-health follow-up workflows, liquid-biopsy interpretation, or epigenetic biomarker analysis, thereby preserving broader cervical and breast cancer pathway value while retaining an implementable molecular entry node.

The disclosure is intentionally drafted to support system claims, method claims, and kit claims directed to cartridge architecture, reader mechanics, optical alignment, temperature control, quality control, assay menus, commercialization variants, and selected deployment extensions while preserving a buildable cartridge-reader combination as the hard product nucleus.

### BRIEF DESCRIPTION OF THE DRAWINGS

FIG. 1 is a schematic system overview showing an example disposable cartridge operably coupled to an example reader, processor, and user-interface or communications subsystem that may optionally route results or metadata to network deployment interfaces.

FIG. 2 is a plan view of an example cartridge showing a sample inlet, lysis chamber, reagent region, subtype-specific reaction regions, and waste reservoir.

FIG. 3 is a sectional representation of an example microchannel geometry showing approximately 100 micrometers of width and approximately 50 micrometers of depth.

FIG. 4 is a workflow diagram illustrating sample loading, lysis, transfer, incubation, optical reading, triage output, and optional same-visit follow-up routing.

FIG. 5 is a schematic of an example reader showing excitation, filtering, detection, heating, sensing, processing, and optional communications components.

FIG. 6 is a schematic multiplex layout showing HPV16, HPV18, pooled high-risk HPV, and internal-control regions.

FIG. 7 is a decision diagram illustrating example control verification, invalid suppression, repeat-test logic, and same-visit triage logic.

FIG. 8 is a kit configuration showing cartridges, swabs, lysis buffer, controls, accessories, and instructions for use in women's health testing workflows.

### DETAILED DESCRIPTION OF THE INVENTION

### Definitions and Claim Construction Guidance

Unless expressly stated otherwise, the terms used herein have the ordinary meanings understood by persons of ordinary skill in the relevant fields of molecular diagnostics, microfluidics, fluorescence measurement, assay design, cartridge manufacturing, and women's health screening. The phrase “high-risk HPV” refers to HPV subtypes associated with clinically significant cervical-cancer risk and includes at least HPV16 and HPV18 and, in some embodiments, one or more additional high-risk classes or grouped targets.

As used herein, “sample-to-answer” refers to a workflow in which an operator introduces a specimen or specimen-derived aliquot into the system and the system produces a readable output without requiring the operator to perform a separate central-laboratory amplification and interpretation process. The phrase does not exclude limited operator steps such as swab agitation, sample transfer, cartridge sealing, or reader insertion.

The word “about” when used with a numerical value encompasses tolerances consistent with intended function, routine manufacturing variation, instrument calibration variation, and ordinary measurement uncertainty. Where a range is stated, endpoints and intervening values are contemplated unless the context clearly requires otherwise. Open-ended terms such as “comprising” and “including” are used in their inclusive sense.

The disclosure is written to support multiple claim classes. Accordingly, no embodiment should be understood as limiting the availability of system, method, cartridge, reader, consumable, or kit claiming unless the language of a later-presented claim expressly so requires. Components described in one embodiment may be combined with features of another embodiment where technically compatible.

### Overall System Architecture

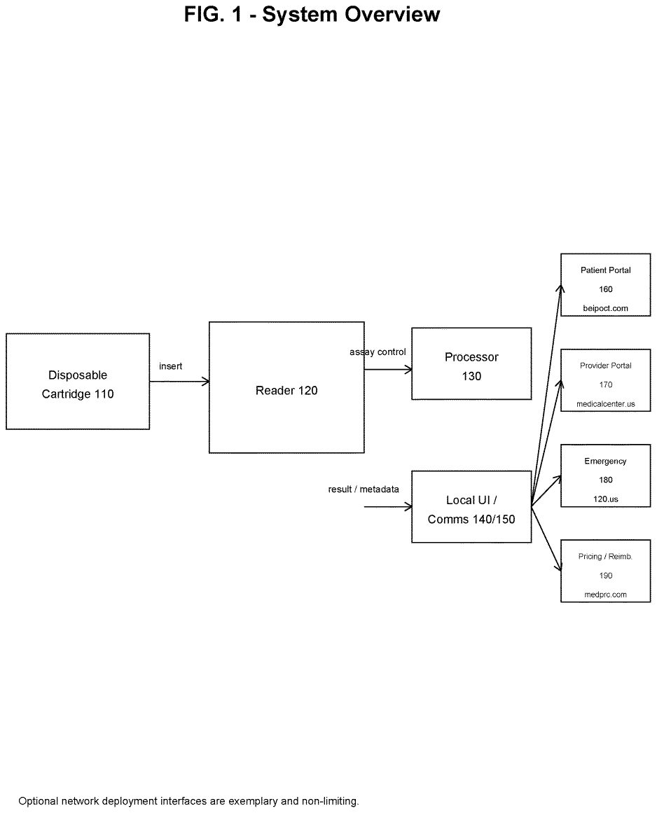

FIG. 1 illustrates an example system 100 including a disposable cartridge 110, a reader 120, a processor 130, and a user interface and communications subsystem 140/150. In representative use, a cervical sample or cervical-sample-derived aliquot is loaded into cartridge 110, the cartridge is inserted into reader 120, and processor 130 coordinates assay timing, optical interrogation, quality-control evaluation, and result issuance.

Cartridge 110 is preferably single-use and configured to contain biological material, lysis constituents, reaction constituents, and waste in a closed or effectively closed fluidic format after sample loading. Reader 120 may be hand-portable, transportable, countertop sized, or integrated into a clinic instrument. In useful embodiments the reader weighs less than five kilograms, is battery-assisted or battery-backed, and is designed for environments where same-visit decision making is beneficial.

Processor 130 may be implemented as a microcontroller, embedded processor, system-on-module, application processor, or functionally equivalent logic unit coupled to memory storing calibration values, assay timing parameters, threshold rules, cartridge lot data, user-interface instructions, and report-generation logic. In some embodiments, processor 130 executes deterministic rules only. In some embodiments, processor 130 additionally performs limited model-assisted signal-quality assessment while preserving the cartridge-centric nature of the invention.

The user interface may include a display integrated into the reader, a simple indicator-panel, a connected smartphone, a tablet, or a remote workstation. The communications subsystem may support wired or wireless transfer of run identifiers, assay-version identifiers, cartridge-lot identifiers, timestamps, and result categories. Such connectivity is optional and should not be read to displace the physical device architecture that anchors the invention.

The system is deliberately modular. A manufacturer may vary enclosure styling, software integration, power source, or external user-interface choices without changing the essential cartridge-reader combination. This modularity is commercially important because it allows private labeling, region-specific product variants, and staged commercialization without requiring abandonment of the structural core disclosed herein.

In one class of embodiments, the system is deployed in an outpatient gynecology clinic or women's health practice. In another class of embodiments, the system is deployed in a mobile van, community screening location, or pharmacy. In another class of embodiments, the system is used in decentralized screening campaigns where immediate triage or referral planning is desirable. These deployment contexts illustrate the practical relevance of the architecture but do not limit the system to any one care setting.

### Cartridge Architecture and Physical Layout

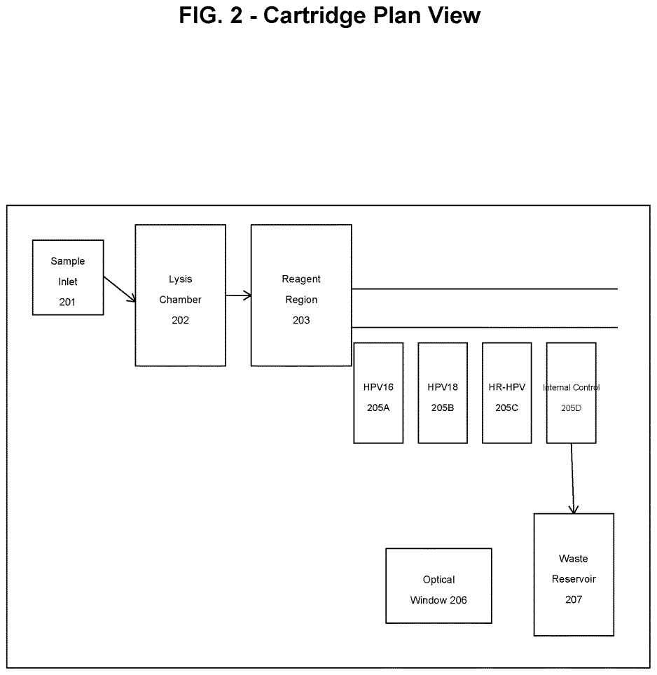

FIG. 2 illustrates an example cartridge plan view. Cartridge 110 may include sample inlet 201, lysis chamber 202, reagent chamber 203, one or more microchannels 204, reaction regions 205A-205D, optical window 206, and waste reservoir 207. The cartridge preferably includes rigid or semi-rigid upper and lower layers defining the fluidic pathway, along with seals, bonds, or equivalent closures selected to preserve fluid integrity during sample loading, incubation, and readout.

In some embodiments, the cartridge body is rectangular and sized for hand loading into a reader bay. In some embodiments, the cartridge body includes alignment rails, tabs, recesses, stops, latch surfaces, or detents that ensure repeatable seating within the reader. In some embodiments, the cartridge includes a keyed asymmetry so that reverse insertion is physically blocked or detected by the reader.

In useful embodiments, the platform is implemented as a cartridge-and-reader system rather than a strip-only assay. The disposable cartridge may be generally rectangular and sized for hand loading into a reader bay, and may include alignment rails, tabs, recesses, stops, or keyed asymmetry that support repeatable seating, optical alignment, and contamination-conscious handling while preserving a compact form factor suitable for clinic, mobile, or supervised near-patient use.

The optical detection zone is preferably positioned relative to a reader optical stack with sufficient repeatability to limit alignment error. In some embodiments, one or more optically transparent windows are formed in the cartridge body or top film. In some embodiments, the read window is locally thinned, polished, planarized, or otherwise configured to minimize optical distortion and autofluorescence.

Waste reservoir 207 preferably provides sufficient downstream containment to prevent backflow, overflow, or release of used assay material. In some embodiments, the waste zone includes absorbent material, gel, porous retention features, dead-end chambers, capillary traps, or volume-expansion regions. In some embodiments, the waste region is permanently sealed once the cartridge is assembled. In some embodiments, the reader compresses or engages a sealing region to improve leak resistance during operation.

The cartridge may include machine-readable identifiers such as barcodes, matrix codes, RFID tags, passive optical fiducials, magnetic markers, or molded lot-indicia. Such identifiers may be read by the reader to load assay menus, calibration constants, expiration data, quality-control thresholds, or operator prompts. The identification features also improve supply-chain traceability and commercial deployment.

Although the figures show a particular layout, other topologies are also contemplated. A cartridge may use a linear flow path, branched paths, radially distributed reaction areas, layered vertical fluid transfer, serpentine channels, or arrays of wells. What is preferred is not a single artistic arrangement, but a cartridge format that preserves reproducible sample transfer, closed handling, controlled reaction conditions, and optically accessible signal generation.

### Cartridge Materials, Surface Treatments, and Assembly

Suitable cartridge substrates include cyclic olefin copolymer, cyclic olefin polymer, polycarbonate, polymethyl methacrylate, polyester laminates, laminates combining rigid and flexible layers, and other materials selected for optical clarity, manufacturability, biocompatibility, chemical resistance, dimensional stability, and cost. In one useful embodiment, a cyclic olefin copolymer base is combined with a bonded cover film providing optical access and fluid containment.

Fabrication methods may include injection molding, hot embossing, roll embossing, laser micromachining, CNC micromachining, lamination, thermal bonding, ultrasonic welding, adhesive lamination, or combinations thereof. Different fabrication methods may be selected for prototype, pilot, and mass-production stages. The disclosure supports each stage because transaction value often depends on a smooth path from concept article to commercial article.

In some embodiments, selected surfaces are treated to improve wettability, capillary transport, reagent compatibility, or anti-fouling behavior. Suitable treatments include plasma treatment, corona treatment, hydrophilic coatings, silanization, grafted surface polymers, blocked reactive coatings, or patterned wetting regions. In some embodiments, hydrophobic barriers or burst valves are used to sequence flow. In some embodiments, both hydrophilic and hydrophobic regions are used in the same cartridge.

Assembly may be carried out in a manner compatible with reagent loading and contamination control. For example, a base layer can be molded or embossed, dried reagents can be deposited into selected chambers, a cover layer can be aligned and bonded, and the completed cartridge can be pouched with desiccant under controlled atmosphere. In other embodiments, wet reagents are held in blister packs or frangible compartments integrated into the cartridge and opened at time of use.

Commercially useful embodiments benefit from dimensional control. For that reason, the disclosure contemplates tolerance management for key features such as channel width, channel depth, optical window planarity, sample-inlet geometry, docking features, and seal thickness. Representative tolerance bands may be selected according to manufacturing capability and assay sensitivity. The specification of preferred feature relationships supports stronger claim drafting and more predictable licensing discussions.

### Sample Collection, Input, and Pre-analytical Handling

A cervical sample may be obtained using a swab, brush, spatula, preservative-based collection device, or functionally similar collection article. In some workflows the specimen is eluted into a liquid medium before transfer to the cartridge. In other workflows the specimen is combined with lysis buffer and then introduced into the cartridge. In other workflows a collection device docks directly with an inlet structure of the cartridge.

The sample inlet preferably accommodates user operation without requiring high precision. In one embodiment, a pipette-deliverable inlet receives a measured aliquot. In another embodiment, the inlet accepts a disposable dropper, transfer bulb, or pre-filled vial nozzle. In another embodiment, the cartridge is coupled to a small sample-preparation tube such that the sample enters when the user actuates a valve or presses the tube into a mating feature.

Pre-analytical handling may include agitation, elution, dilution, viscosity adjustment, debris management, or preservative neutralization. In some embodiments, the lysis zone contains chemistry sufficient to handle preservative-containing samples. In some embodiments, the cartridge includes one or more filters, membranes, inert beads, or passive separation structures to reduce fibers, mucus, large particulates, or bubbles before the sample reaches a reaction zone or optical zone.

The invention contemplates clinical workflows with minimal operator burden. In one workflow, a swab is eluted in lysis buffer, a measured aliquot is transferred to the cartridge, the inlet is closed, and the cartridge is inserted into the reader. In another workflow, the sample is introduced directly and the reader triggers reagent release or transfer steps after cartridge insertion. The detailed support for these workflows strengthens utility across different product-cost targets.

The system is preferably tolerant to routine variation in collection quality while still maintaining quality-control safeguards. Accordingly, some embodiments include sample sufficiency controls, fluid-transfer controls, and internal controls that collectively reduce the risk of false negative, false positive, or non-actionable outputs. Where the input quality is too poor for reliable interpretation, the system may issue an invalid or repeat-test result rather than a potentially misleading positive or negative call.

### Lysis Chemistry and Sample Conditioning

The lysis zone or coupled lysis chamber may contain one or more detergents, chaotropes, salts, buffering agents, chelators, enzymes, or stabilizers selected to release nucleic acid targets compatible with downstream CRISPR reaction chemistry. Suitable lysis strategies may be optimized for speed, shelf life, simplicity, inhibition control, and compatibility with portable deployment.

In one embodiment, lysis occurs on-chip in a chamber upstream of the reaction region. In another embodiment, lysis occurs in a small off-chip vessel fluidically coupled to the cartridge. In another embodiment, the reader mechanically ruptures a blister or frangible compartment that releases lysis solution into the loaded sample after cartridge insertion. In each case, the design objective is to keep the workflow compact and reproducible.

Sample conditioning may include pH adjustment, ionic-strength adjustment, release of RNase inhibitors, stabilization of reporters or enzymes, or dilution of inhibitors present in the sample medium. In some embodiments, a transfer segment between the lysis region and reaction region includes passive mixing structures, fluidic delay features, or dissolvable barriers that provide a defined dwell time before the sample enters the reaction channel.

Some embodiments emphasize minimal user steps and low manufacturing complexity; others emphasize integrated automation. For example, one embodiment may use a two-step manual transfer into a sealed cartridge, whereas another embodiment may use reader-actuated reagent release and timed transfer to support a premium integrated consumable. Both are commercially meaningful embodiments and both remain within the scope of a cartridge-reader point-of-care invention.

The lysis chemistry and sample-conditioning features are particularly valuable because they support performance consistency in decentralized settings. Many transaction counterparties focus not merely on the detection chemistry, but on whether the product can actually handle real specimens without requiring a central laboratory. The present disclosure therefore provides detailed support for embodiments that integrate pre-analytical handling into the cartridge workflow.

### Microchannel Geometry and Fluidic Design

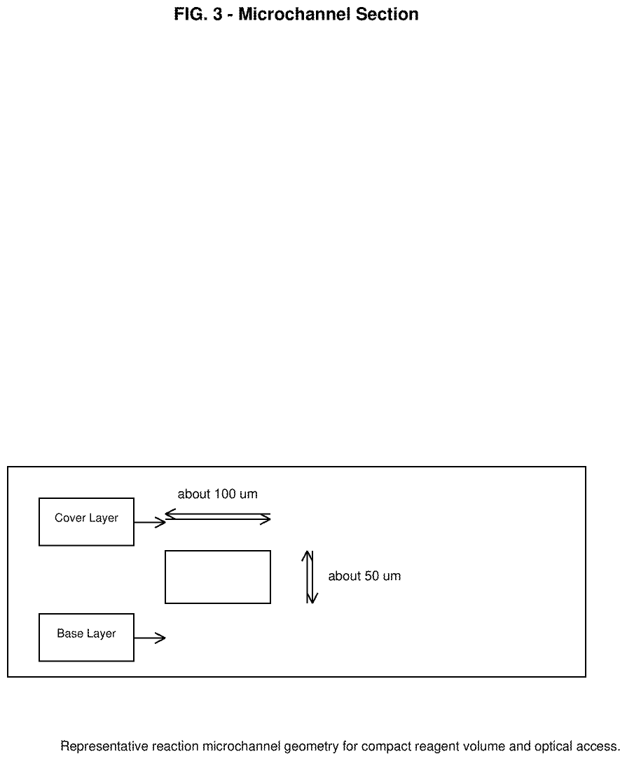

FIG. 3 depicts an example microchannel profile in which a principal reaction microchannel has a width of about 100 micrometers and a depth of about 50 micrometers. This geometry is useful because it balances reagent economy, fluid-transfer predictability, reaction-volume control, optical accessibility, and manufacturability in compact polymer-based cartridges.

The invention is not limited to a single exact geometry. In some embodiments, useful channel widths fall in a range of about 80 to about 150 micrometers. In some embodiments, useful channel depths fall in a range of about 30 to about 80 micrometers. In some embodiments, the principal channel is approximately 100 by 50 micrometers, which can provide a particularly effective compromise among capillary behavior, diffusion distance, reaction speed, and optical signal acquisition.

Channels may be rectangular, trapezoidal, rounded, semi-rounded, or otherwise shaped, provided they preserve predictable transfer and readable optical response. In some embodiments, a main channel feeds multiple subtype-specific reaction chambers. In some embodiments, a trunk channel distributes fluid to parallel arms. In some embodiments, each subtype region includes a local expansion chamber or read cavity sized to improve optical path stability.

Fluid propulsion may be passive, capillary-assisted, pressure-assisted, vacuum-assisted, mechanically actuated, centrifugally assisted, or reader-actuated. In low-complexity embodiments, capillary forces and valve structures are preferred. In higher-control embodiments, the reader may apply pressure, vacuum, or mechanical force to move the sample or reagents between compartments. The disclosure supports these alternatives because market segments differ in acceptable instrument complexity.

Additional microfluidic features may include bubble traps, vent channels, dead-volume minimization structures, passive mixers, dissolvable films, burst valves, capillary stop valves, siphons, or flow-resistor elements. These features improve sequence control, reduce air-related signal artifacts, and support reproducible arrival of conditioned sample into the reaction zone. Their inclusion in the specification increases support for future claim differentiation if specific prior art appears during prosecution.

In some embodiments, different channels or zones share a common cross-sectional design to simplify tooling. In other embodiments, reaction channels, waste conduits, and priming conduits have distinct geometries matched to their functions. A premium product variant may therefore use differentiated fluidic architecture, whereas a cost-sensitive version may rely on a more uniform channel family. Both approaches are contemplated.

### Reaction-Zone Layout and Multiplex Architecture

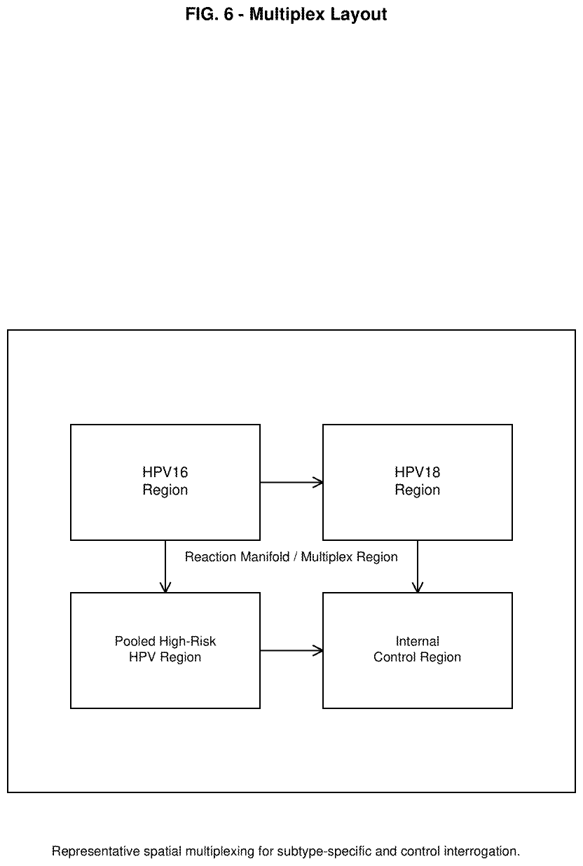

FIG. 6 illustrates a multiplex layout in which separate reaction regions correspond to HPV16, HPV18, pooled high-risk HPV, and internal control. Spatial separation of these regions is particularly useful because it simplifies optical decoding, supports intuitive interpretation, and provides tangible structural anchors for system and cartridge claims.

In one embodiment, each region contains one or more guide RNAs and corresponding reporter systems tuned to the intended target. In another embodiment, grouped targets are detected using pooled guide sets, with one region dedicated to a broader high-risk HPV class rather than a single genotype. In another embodiment, one or more duplicate or verification regions are included to improve confidence or support premium assay versions.

The internal-control region may verify assay chemistry, fluid transfer, optical function, sample adequacy, or one or more combinations thereof. In some embodiments, the control region responds to a non-target control template or a defined internal-control chemistry. In some embodiments, the control region is configured to fail visibly or algorithmically when lysis, transfer, reaction, or readout has been materially compromised.

Reaction regions may be spatially separated by walls, hydrophobic boundaries, fluidic valves, discrete wells, or channel branches. The regions may be interrogated sequentially, simultaneously, or in a hybrid timing sequence. Spatial multiplexing is preferred in many embodiments because it simplifies small-format optics, but temporal and spectral multiplexing options are also contemplated where commercially justified.

A cartridge family may support region-specific product variants. For example, one product variant may include only HPV16, HPV18, and pooled high-risk HPV regions. Another may add confirmatory regions, contamination-check regions, or region-specific localization markers. Another may substitute a different women's health target panel while preserving the same docking and optical interfaces. The present disclosure is drafted to support that product-family logic without abandoning the primary HPV embodiment.

### CRISPR-Cas13a Assay Chemistry

The reaction zones preferably contain a CRISPR-Cas13a chemistry capable of producing target-dependent reporter cleavage and fluorescence generation. A suitable reaction mixture may include Cas13a enzyme, one or more guide RNAs, one or more reporter molecules, buffering agents, salts, stabilizers, and optional additives selected to support isothermal operation in a compact cartridge environment.

In useful embodiments, the reaction is conducted at about 37 degrees Celsius for about 15 minutes. This combination is particularly attractive because it supports compact thermal design, relatively low power consumption, and a clinically relevant same-visit turnaround. The disclosure also contemplates adjacent ranges and alternative dwell times where reagent or target characteristics warrant adjustment.

Guide RNA design may be configured for genotype-specific recognition or grouped recognition. For example, one reaction region may contain guide material for HPV16, a second for HPV18, and a third for a pooled set of other high-risk HPV classes. The design rules for such guides may take into account target conservation, mismatch tolerance, cross-reactivity avoidance, secondary structure, reagent stability, and manufacturing practicality.

Some embodiments use amplification-free detection. Other embodiments incorporate target pre-amplification or upstream enrichment compatible with a point-of-care format. The invention is anchored not to any one purely academic reaction diagram, but to a cartridge-reader implementation in which the molecular events are integrated into a commercially practical pathway. As a result, the specification supports both immediate POCT embodiments and later optimized chemistry embodiments.

Reagents may be stored dry within chambers, dried on reaction surfaces, held in frangible ampoules, packaged in blister reservoirs, or supplied partly on-cartridge and partly off-cartridge. Reagent stabilization strategies may include sugar matrices, polymer stabilizers, lyophilization-compatible excipients, moisture barriers, oxygen barriers, and temperature-managed packaging. Such details are commercially significant because field deployment frequently depends on shelf life and transport tolerance.

Control chemistry may be included to detect inhibition, failed transfer, failed rehydration, reader malfunction, or reagent degradation. In some embodiments, the control chemistry is positive-control-like. In other embodiments, it is process-control-like. In yet other embodiments, different controls are distributed across different reaction regions. The present disclosure supports each approach because robust control behavior materially affects both regulatory credibility and transaction value.

### Reporter Architecture and Quantum-Dot Fluorescence

In preferred embodiments, reporter activation is read through quantum-dot-enhanced fluorescence. In one especially useful embodiment, the reporter exhibits a peak emission at about 605 nanometers. This wavelength is advantageous because it permits a compact optical stack, supports stable discrimination in a small reader, and aligns with a practical balance of signal brightness and reader simplicity.

Quantum dots may be used directly or as part of a reporter system that couples target recognition to fluorescent change. The disclosure is not limited to a single chemistry pathway for that coupling. Rather, it supports reporter architectures in which target-dependent activity produces a measurable optical change that can be detected through a defined read window in a compact instrument.

In some embodiments, the optical readout is single-color and intentionally simplified to maximize robustness, manufacturability, and sensitivity in a compact reader. In some embodiments, multi-color or reference-channel approaches are used to support normalization, additional controls, or expanded multiplexing. The single-color 605-nanometer embodiment is nonetheless particularly valuable as a practical commercial baseline.

The optical window, reporter concentration, detector selection, excitation wavelength, filtering characteristics, and signal-integration period may be jointly optimized to maximize signal-to-background ratio. In some embodiments, one or more reference features are used to normalize manufacturing variation, ambient variation, or reader-to-reader variation. Such normalization features are useful not only technically, but also commercially, because they can reduce field support burden and lot-to-lot variability.

The specification deliberately describes the optical strategy as part of a whole product, not as a detached theoretical choice. A fluorescence-reporting approach that can be aligned with a cartridge bay, repeatably interrogated, and converted into a triage output contributes directly to the patent asset's licensing relevance. This is especially true where the optical design cooperates with the reaction geometry and quality-control logic described elsewhere in the disclosure.

### Reader Mechanical Docking and Cartridge Handling

Reader 120 preferably includes a docking bay configured to receive cartridge 110 in a controlled orientation. In some embodiments, the bay includes rails, guides, latch elements, spring contacts, sensors, stops, or mechanical references that ensure repeatable positioning of the cartridge relative to the heater and optical path. In some embodiments, insertion is manual and full seating is confirmed by a switch or optical detector. In other embodiments, a motorized mechanism assists closure or engagement.

The docking architecture may also perform protective functions. For example, the reader may shield the optical window from ambient light, provide local thermal insulation around the reaction zone, isolate the cartridge from operator touch during reading, or apply local pressure to improve thermal contact and fluidic stability. In some embodiments, the reader includes a bay door or lid that both blocks light and serves as an interlock.

In field-friendly embodiments, the docking process is tolerant to inexperienced users. The cartridge can be inserted in a single gross motion, after which the instrument confirms presence, reads identifiers, initiates a preheat sequence, and guides the user through the next step if any further step is needed. The resulting ease of use is important to same-visit deployment and therefore to the commercial strength of the patent asset.

Mechanical features that cooperate with the cartridge may support premium product variants. For example, the reader may include an actuator that ruptures reagent blisters, a compression member that improves seal integrity, or a thermal interface pad that reduces gradients across the reaction zone. These variants are disclosed because they may become useful claim-differentiation tools if competitor products emphasize instrument-assisted consumable actuation.

The docking and handling features additionally support a standalone reader claim or continuation strategy in which the reader is separately claimed as a cartridge-processing instrument. The present specification therefore provides enough mechanical context to support such claim families while keeping the present application focused on the core integrated product concept.

### Thermal Control Subsystem

Reader 120 preferably includes a temperature-control subsystem configured to maintain the reaction at about 37 degrees Celsius within an acceptable tolerance band. Suitable components include resistive heaters, thin-film heaters, Peltier elements, conductive blocks, thermal interface layers, temperature sensors, and corresponding control electronics. The selected implementation may vary according to product cost and deployment environment.

In one embodiment, a heater is positioned beneath or adjacent the cartridge reaction zone and is controlled by a feedback loop using one or more temperature sensors. In another embodiment, multiple sensors are used to estimate local gradient or compensate for environmental variation. In another embodiment, one sensor is associated with the heater assembly and another sensor is associated with the cartridge bay or ambient environment.

Control algorithms may include PID control, threshold control, duty-cycled control, feed-forward compensation, or hybrid control strategies. The processor may use stored calibration data or cartridge-lot metadata to adjust control behavior. In some embodiments, the reader performs a pre-equilibration interval before initiating the reaction timing window. In some embodiments, reaction timing begins only after a target temperature band is reached and maintained for a minimum dwell period.

Thermal design considerations include warm-up time, power draw, hotspot reduction, insulation, condensation control, and compatibility with the cartridge materials. A field-deployable embodiment may prioritize battery efficiency and rapid stabilization, whereas a benchtop embodiment may prioritize multi-run repeatability. The specification supports both because transaction and licensing potential often depend on adaptability across deployment tiers.

A preferred commercial variant is one in which the thermal subsystem is strong enough to support a same-visit workflow without overcomplicating the reader. That balance contributes to the practical identity of the invention: a real product module rather than a laboratory-only experiment. Accordingly, the disclosure gives thermal control a prominent role in the integrated architecture.

### Optical Excitation, Detection, and Signal Acquisition

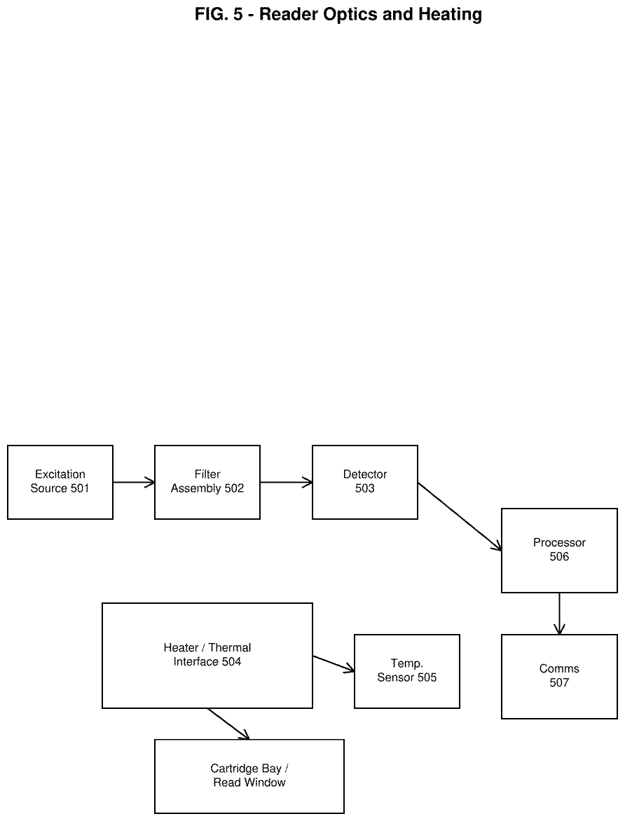

FIG. 5 illustrates a representative optical subsystem including excitation source 501, filter assembly 502, detector 503, heater 504, temperature sensor 505, and processor 506. The excitation source may include a light-emitting diode, laser diode, vertical-cavity source, or functionally similar light source selected for compatibility with the reporter chemistry. The detector may include a photodiode, CMOS sensor, CCD sensor, silicon photomultiplier, avalanche detector, or equivalent sensor.

Filter assembly 502 may include one or more excitation filters, emission filters, dichroic elements, apertures, lens elements, reflective structures, or molded optical guides. In some embodiments, the optical path is intentionally simple to reduce cost and improve ruggedness. In other embodiments, additional optical elements are added to increase sensitivity, reject ambient light, or permit reference-channel correction.

Signal acquisition may involve one or more integration periods, background sampling, dark-signal subtraction, baseline estimation, temporal averaging, or normalization steps. In some embodiments, the reader acquires control-region and test-region signals in a defined order and applies calibration or confidence rules before returning a result. In some embodiments, repeated readings are taken over time to estimate slope, plateau, or endpoint behavior.

The optical geometry is preferably coordinated with cartridge design. Read windows, channel depth, reporter concentration, and excitation intensity can all influence measured signal. The disclosed specification therefore describes these features together rather than in isolation, because the value of the patent asset lies in the integrated product logic. A competitor seeking to reproduce the commercial advantages of the system would need to consider this interaction among geometry, chemistry, and optics.

In some embodiments, the reader performs a self-check or reference check before or during a run. This may include reading a known reference region, verifying detector response, or compensating for temperature-dependent optical drift. Inclusion of such features further supports quality-control dependent claims and strengthens the story for licensing into regulated or semi-regulated product lines.

### Electronics, Firmware, and User Interface

Processor 130 may cooperate with memory storing firmware, assay definitions, timing parameters, calibration data, lot-specific thresholds, quality-control logic, event logs, and user-interface content. In some embodiments, memory is nonvolatile and permits traceability across multiple runs. In some embodiments, service or diagnostic partitions are also included. The resulting reader is therefore not merely a light source and detector but an integrated assay appliance.

Firmware may manage cartridge detection, lot identification, thermal ramping, timing windows, optical sampling, result computation, event logging, error handling, and display behavior. In some embodiments, firmware updates permit controlled expansion of supported assay menus while preserving the same hardware platform. Such upgradability is commercially meaningful because it allows portfolio growth without sacrificing the cartridge-reader core.

The user interface may be minimal or rich. A low-cost version may use indicator lights or a simple display, whereas a richer version may use a touch screen or mobile-app interface. In some embodiments, the displayed information includes assay countdown, cartridge status, invalid warnings, lot mismatch warnings, quality-control pass/fail status, and final triage category. In some embodiments, role-based displays differentiate between operator and supervisor information.

Event logging is valuable for both product support and transaction value. The reader may store timestamps, cartridge identifiers, result category, user actions, error codes, firmware versions, assay versions, and maintenance history. Such records can support field troubleshooting, quality systems, regulatory inspections, and diligence review in licensing or assignment transactions. The specification includes these details because they are part of what makes the invention commercially serious.

Connectivity may be wired or wireless and may support result export, audit transfer, maintenance diagnostics, or software configuration. However, the disclosure does not depend on connectivity for patentability. Rather, communications capability is presented as an optional layer that enhances deployment flexibility without displacing the device-centered character of the invention.

### Quality Control, Calibration, and Invalid Suppression

Quality control is a preferred differentiator of the present invention. The processor may verify that one or more control criteria are satisfied before issuing a final result. Such criteria may include internal-control signal above threshold, background below threshold, acceptable ratio between test and reference regions, acceptable timing profile, acceptable cartridge-identification match, or combinations of these.

In one useful embodiment, the system suppresses result issuance when an internal control fails a predetermined acceptance criterion. The system may instead output “invalid,” “repeat,” or a similarly non-affirmative message. This protects patient safety, improves credibility in decentralized settings, and increases the value of the platform to transaction counterparties concerned about field robustness.

Calibration may be reader-specific, lot-specific, assay-version-specific, or environment-aware. For example, a cartridge lot may be associated with one or more calibration constants read from a code or stored in the reader memory. In some embodiments, the reader applies a baseline-correction factor, temperature-compensation factor, or threshold table associated with the lot or assay version. In some embodiments, calibration is supplemented by an optical reference structure on the cartridge.

The disclosure also supports premium embodiments in which confidence scoring or model-assisted quality assessment is used as a secondary safeguard. Such assessment may analyze signal shape, region-to-region consistency, or error signatures. Even in such embodiments, the claimed invention remains centered on the physical cartridge-reader architecture and its assay workflow rather than on an abstract software concept.

Quality-control dependent behavior may influence triage output. For example, a test may be categorized as invalid when the control is absent, as repeat-test recommended when signal-to-noise is borderline, or as a normal triage category when both control and analytical thresholds are satisfied. The detailed support for these distinctions strengthens prosecution flexibility and later commercial positioning.

In some embodiments, service diagnostics are separated from assay quality control. For example, the reader may detect heater drift or optical degradation and flag the instrument for service, while still separately evaluating whether a given cartridge run is analytically valid. This distinction is useful in larger installed fleets and therefore supports the system's value as a scalable platform.

### Triage Logic and Result Categories

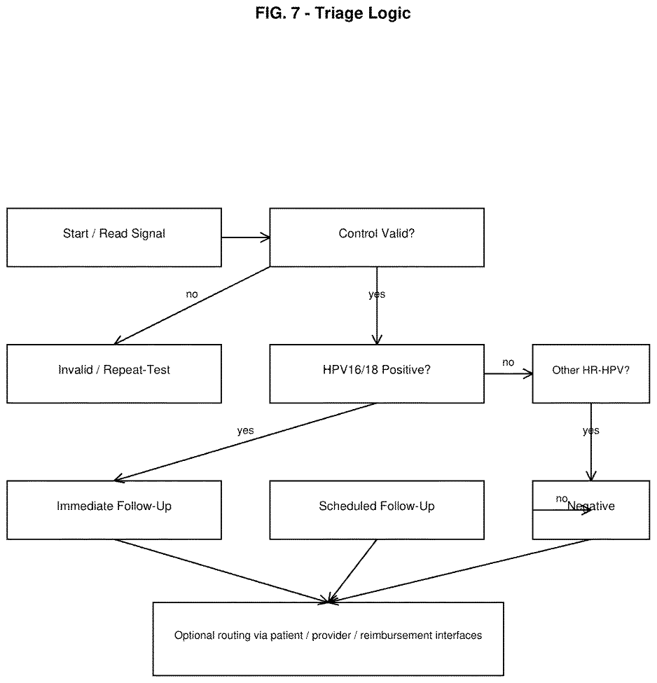

FIG. 7 depicts an example decision pathway. In one preferred embodiment, the processor classifies the run into one of four categories: HPV16/18 positive, other high-risk HPV positive, negative, or invalid. These categories are clinically intuitive and commercially attractive because they support same-visit counseling while remaining narrow enough for a compact assay menu.

Result categories may be generated through deterministic rule sets that incorporate target-region signal, control-region signal, baseline adjustment, and threshold comparison. In some embodiments, a positive result is assigned when a region-specific signal exceeds a threshold and control requirements are met. In some embodiments, different thresholds or decision windows are used for genotype-specific versus grouped-target regions.

The system may optionally generate workflow prompts associated with the result. A positive HPV16/18 result may trigger a message recommending immediate follow-up under the relevant clinical program. Another high-risk HPV positive result may trigger a different message or interval suggestion. A negative result may trigger routine follow-up messaging. An invalid result may instruct repeat sampling or repeat testing. These prompts may be tailored by locale or customer without altering the cartridge architecture.

Same-visit triage is especially useful because a patient can leave the encounter with a clearer next step. The disclosed system therefore focuses not merely on laboratory classification, but on practical result packaging. This feature also increases transaction value because a buyer or licensee can more readily imagine integration into clinical workflows rather than treating the invention as a raw assay concept only.

In some embodiments, the reader stores both a simplified result category and supporting metadata such as confidence values, raw channel intensities, or event codes. A regulatory or supervisory user may therefore access more detail than a routine operator, while the ordinary user sees a concise actionable output. Such multi-layer output design is commercially attractive and is fully supported by the present disclosure.

### Workflow Embodiments

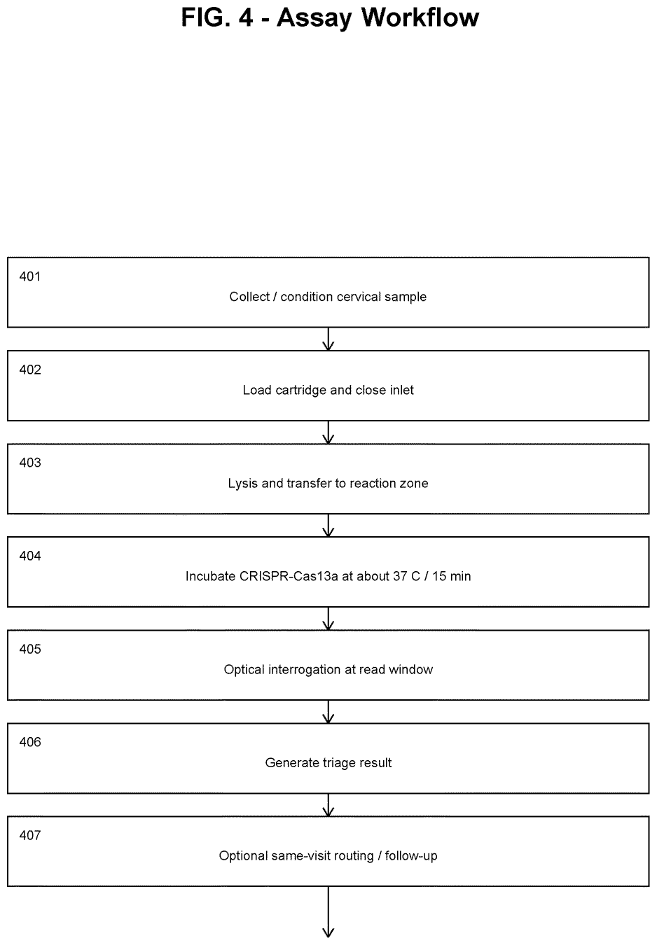

FIG. 4 depicts an example workflow in which a user receives a cervical sample, loads a conditioned aliquot into the cartridge, initiates lysis and transfer, inserts the cartridge into the reader, and starts the run. The reader then controls incubation, acquires fluorescence, evaluates control status, and outputs a triage result. In a representative embodiment, hands-on time is under about five minutes and the molecular reaction period is about fifteen minutes, allowing a same-visit workflow.

In representative use, a cervical sample or cervical-sample-derived aliquot is introduced into the disposable microfluidic cartridge through the sample inlet. The cartridge contains a lysis zone, one or more reaction regions, an optical detection zone, and a waste reservoir in a closed or effectively closed fluidic format. After loading, the cartridge is inserted into the reader, which controls assay timing and temperature, including incubation of a CRISPR-Cas13a reaction at about 37 degrees Celsius for about 15 minutes, and optically interrogates fluorescence associated with the reaction. The processor evaluates control criteria and converts the measured signal into a same-visit triage result, which may include HPV16/18 positive, other high-risk HPV positive, negative, invalid, or repeat-test recommended.

A lower-complexity workflow may use manual lysis and transfer with a low-cost cartridge. A higher-integration workflow may use reagent blisters, reader-actuated sequencing, and automatic timing. A public-health workflow may use barcoded cartridges and central result export. A physician-office workflow may prioritize simple in-room operation and immediate patient counseling. The same core cartridge-reader concept can support each of these variants.

In some embodiments, the system is used in scheduled screening. In some embodiments, it is used for opportunistic testing during a routine clinical visit. In some embodiments, it is deployed for mobile outreach or rural screening. In some embodiments, it is used in workplace or community events. The disclosure includes these examples not to broaden the invention into abstraction, but to demonstrate that the cartridge-reader module has practical deployment relevance.

Workflow design may also account for operator training. Step ordering, prompts, interlocks, and cartridge coding may all be selected to reduce confusion and user error. For example, the reader may refuse to start a run if the cartridge code is unreadable, expired, or mismatched. Such operational details matter in real product deployment and therefore contribute to the patent asset's value.

The same-visit emphasis is particularly important. Many women's health programs lose value when results arrive long after collection because follow-up becomes harder. The disclosed architecture addresses this by combining a point-of-care consumable with an instrument that can produce a clinically useful categorized result in a compact time window. That product logic is central to the invention's identity and transaction attractiveness.

### Reader Data Logging, Auditability, and Export

In some embodiments, the reader stores a run log including date, time, cartridge lot, assay version, firmware version, user identifier, result category, and selected quality-control metrics. This information can be exported, archived, or reviewed by supervisors. Such features are valuable in regulated or quality-managed environments and can also improve supportability of decentralized deployments.

Export formats may include encrypted files, secure wireless transfer, wired transfer, or structured reports suitable for clinic records. In some embodiments, only summarized results are exported. In other embodiments, summarized results and selected metadata are exported. In still other embodiments, raw data or compressed raw data are also available for research or advanced troubleshooting under suitable controls.

The inclusion of logging and export features improves licensing value because it demonstrates that the platform has been considered as a real product system. Potential transaction counterparties frequently examine whether an invention can fit into quality systems, service models, and post-market support workflows. The present disclosure accordingly supports not just a chemical assay, but a supportable instrumented product.

Auditability can also support fleet management. Service intervals, performance flags, and event histories may be tracked across multiple readers. In some embodiments, a supervisory interface or service tool can extract maintenance records. This information is commercially useful in organizations deploying readers across multiple clinics or screening sites.

### Controls, Calibrators, and Reference Articles

The invention may be practiced with positive controls, negative controls, process controls, optical references, temperature references, and lot calibrators. Such materials may be supplied as separate articles in a kit or integrated into the cartridge architecture. Positive controls may verify that one or more targeted reaction pathways remain functional. Negative controls may detect contamination or background drift. Process controls may verify sample handling and chemistry performance.

In some embodiments, a calibration cartridge is provided separately from test cartridges. A service technician or supervisor may insert the calibration cartridge into the reader to verify optics, heating, and signal thresholds. In some embodiments, a packaging label or memory element stores the calibration data applied to a production lot. In some embodiments, calibration is refreshed by software while preserving the same hardware architecture.

Reference articles may also be used in manufacturing release, stability testing, field support, or operator training. Because these reference articles interact directly with the reader and the cartridge ecosystem, the present disclosure supports follow-on claiming directed to such commercial accessories where strategically desirable.

The inclusion of controls and calibrators is not merely a regulatory afterthought. It is a structural part of making the invention useful, credible, and tradeable. A party considering license, acquisition, or integration into a product family will often value the system more highly when the specification demonstrates awareness of calibration and quality infrastructure. For this reason, the present specification gives them explicit treatment.

### Manufacturing and Scaled Production Embodiments

In one production pathway, a polymer substrate is formed by injection molding with optical and docking features molded in the same tool set. Reaction chambers and channels are subsequently populated with dried reagents, after which a cover layer is aligned and bonded. The completed cartridge is then pouched with desiccant and labeled with machine-readable lot information. This pathway is well suited to moderate-and high-volume manufacturing.

In another production pathway, early-stage or specialty cartridges are produced by micromachining or laser patterning followed by lamination. This can be useful during prototype optimization, low-volume pilot runs, or customized panel development. The disclosure supports this variant because high-value patent assets frequently begin in lower-volume development settings before transitioning to mass manufacture.

Reader manufacture may use a modular architecture in which optics, heater assembly, control electronics, user interface, and enclosure are assembled as submodules. Such modularity can reduce service complexity and support product differentiation. A premium reader may include richer connectivity, expanded storage, or enhanced calibration features, while an entry-level reader may retain only the functions necessary for the core assay.

The specification also contemplates statistical process control, lot release testing, seal integrity testing, and optical-window inspection as part of scaled manufacturing. These details support the practical image of the invention as a real commercial system and create additional material for later claim differentiation or diligence support.

Manufacturing strategy matters to value. A patent asset that clearly contemplates prototype build, pilot production, and mass production is generally easier to position in licensing discussions than one that speaks only at a conceptual level. The present disclosure therefore embeds manufacturing logic throughout the description.

### Packaging, Shelf Life, and Distribution

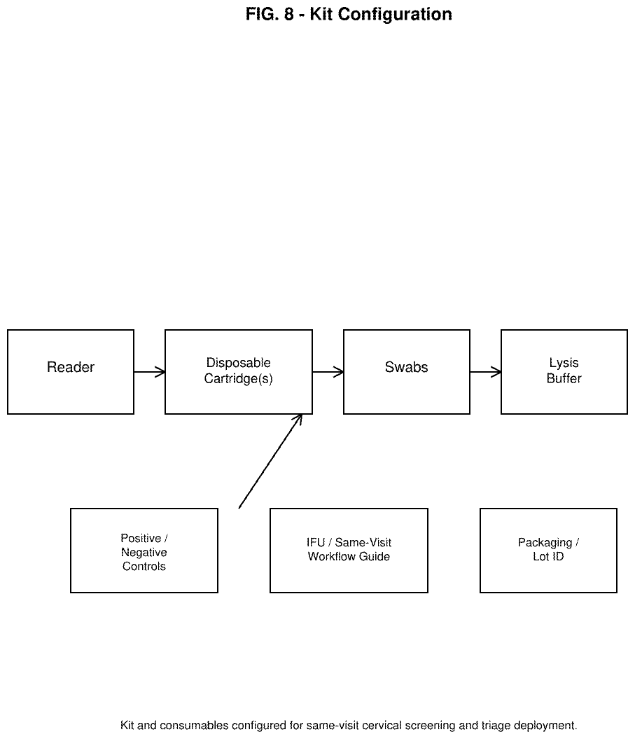

FIG. 8 shows a kit configuration including cartridges, swabs, lysis buffer, controls, accessories, and instructions for use. Cartridges may be individually pouched, nested in trays, supplied in lot-organized cartons, or packaged in other formats suited to transport, storage, and field deployment. Desiccants, foil barriers, inert atmospheres, humidity indicators, or tamper features may be used to improve shelf life and quality assurance.

Reagents may be stored dry within the cartridge, within sealed reagent compartments, or in separate auxiliary containers. Different packaging strategies may be selected depending on target market, cold-chain availability, desired shelf life, and acceptable cartridge complexity. A low-resource variant may prioritize room-temperature stability. A premium clinic variant may prioritize integrated automation even if the cartridge is more complex.

The kit may also include cleaning tools, charging accessories, barcode labels, control articles, disposable transfer tools, operator quick-reference cards, or digital instructions. The details of kit composition can materially affect transaction value because they influence distribution cost, regulatory strategy, and partner integration. The present disclosure therefore supports kit-level claiming and related continuation strategies.

Shelf-life engineering may address moisture control, oxygen exposure, light sensitivity, freeze-thaw tolerance, and vibration tolerance. In some embodiments, lot-specific expiration data are machine-readable and can be checked by the reader before a run starts. Such features are practically useful and also reinforce the image of the invention as a product platform that can actually move through commerce.

### Contamination Control and Biosafety

Because point-of-care molecular testing can be sensitive to contamination, the cartridge is preferably configured to limit aerosol formation, backflow, and post-reaction leakage. Sealed loading, unidirectional flow, dead-end waste capture, and controlled venting can all contribute to contamination control. In some embodiments, the cartridge remains closed from the moment of sample loading until disposal. In some embodiments, the reader also provides shielding or containment around the cartridge during the run.

The optical and fluidic layout may reduce contamination risk by minimizing operator interaction after sample introduction. For example, if lysis, transfer, reaction, and readout occur within a closed consumable, the opportunity for cross-contamination is reduced relative to open-well manipulations. This practical advantage strengthens the case for licensing or deployment in decentralized settings.

In some embodiments, surfaces exposed to waste or residual sample are selected for compatibility with decontamination strategies or safe disposal. In some embodiments, the cartridge housing includes visual indicators confirming that the cartridge has been used. In some embodiments, used cartridges are retained within a sealed pouch or caddy after removal from the reader. The disclosure supports such features because biosafety behavior is part of commercial readiness.

Contamination control may also be monitored analytically. For example, a negative-control region or contamination-check region may be used in some product variants. The presence of such support in the specification increases flexibility if prosecution or market analysis later shows value in claims directed to contamination-specific safeguards.

### Alternative Panels and Assay-Family Expansion

Although the presently preferred claims focus on high-risk HPV genotyping and cervical cancer triage, the same cartridge-reader architecture may support related women's health molecular menus. For example, a product family may use the same mechanical cartridge interface and reader optical pathway while substituting different guide sets, controls, or reaction-region patterns to target related analytes relevant to women's health screening.

Such panel expansion may include modified HPV groupings, reflex confirmatory targets, adjunctive infection markers, sample-quality indicators, or future analyte panels compatible with the disclosed fluidic and optical architecture. The present application nonetheless maintains a focused claim posture because the strongest near-term asset often arises from a coherent first product, not from an unfocused catalogue of hypothetical uses.

A portfolio owner may therefore use this specification as a support base for future continuation practice, licensing conversations, or product-roadmap planning. The fact that the architecture can host family expansion without losing its concrete device identity contributes to the invention's long-term strategic value.

### Use Environments and Commercial Deployment

The invention is useful in physician offices, women's health clinics, urgent care sites, community screening events, pharmacies, mobile medical vans, public-health campaigns, and other decentralized testing environments. The compact cartridge-reader format is particularly beneficial where rapid turnaround and practical triage are more valuable than batch laboratory throughput.

In some embodiments, the system is deployed as a stand-alone clinic device. In some embodiments, it is part of a supervised network of instruments operated across multiple sites. In some embodiments, it is used as a partner-integrated module within an established women's health product family. These deployment models matter because they align with real transaction pathways such as licensing, co-branding, or distribution partnerships.

The invention also has value as an asset package because it is not limited to a bare scientific reaction. It includes a consumable architecture, a reader architecture, quality control, calibration concepts, kit support, and deployment logic. That level of integration is attractive in assignment or license discussions because it reduces the amount of conceptual work a counterparty must perform before seeing a plausible commercial path.

At the same time, the present application purposely avoids overloading the core claims with remote portals, reimbursement engines, or unrelated oncology narratives. Those may be pursued elsewhere if strategically useful. Here, the emphasis remains on a patent-ready product nucleus that is easier to examine, easier to position, and more likely to serve as the anchor of a layered portfolio.

### Representative Operating Parameters and Design Ranges

Representative but non-limiting operating parameters include reaction incubation at about 37 degrees Celsius for about 15 minutes, channel geometry of about 100 micrometers width by about 50 micrometers depth, fluorescence reporting near 605 nanometers, and categorized output including HPV16/18 positive, other high-risk HPV positive, negative, or invalid. These features define a particularly attractive commercial embodiment because they cooperate well in a compact, practical instrument.

Other useful embodiments may vary one or more of these parameters while preserving the integrated point-of-care architecture. For example, channel width or depth may vary to fit a chosen manufacturing method or optical geometry, reaction timing may vary to fit reagent chemistry, and output categories may be localized to fit regional practice or partner preference. The disclosure therefore supports both a preferred core embodiment and a commercially adaptable family of close variants.

Importantly, these parameters are presented as parts of a whole. The invention does not rest on a single number detached from context. Rather, the preferred values are included because, taken together with the cartridge-reader architecture and triage logic, they define a concrete and practical product. That integrated disclosure is expected to improve prosecution flexibility and commercial persuasiveness.

### SPECIFIC EXAMPLES

Example 1. A disposable cartridge formed from cyclic olefin copolymer includes a sample inlet, lysis chamber, reaction channel, optical window, and waste reservoir. A cervical sample is introduced, lysed, and transferred through a principal channel of approximately 100 micrometers width and approximately 50 micrometers depth. The cartridge is inserted into a reader that heats the reaction zone to about 37 degrees Celsius and reads fluorescence near 605 nanometers to produce a categorized triage result.

Example 2. A multiplex cartridge includes distinct regions corresponding to HPV16, HPV18, pooled high-risk HPV, and an internal control. The reader interrogates the regions in sequence, verifies that the internal control satisfies an acceptance criterion, and suppresses result issuance if the control fails. If the control passes, the processor returns one of four categories: HPV16/18 positive, other high-risk HPV positive, negative, or invalid.

Example 3. A field-deployable reader uses a battery-backed heater, optical detector, and onboard memory. An operator loads a conditioned sample into a sealed cartridge, inserts the cartridge, and starts a same-visit workflow. Run identifiers, lot information, and result categories are stored locally and can be exported after the run for quality management or clinical records.

Example 4. A premium consumable includes dried CRISPR-Cas13a reagents in separate reaction zones and a blister-actuated lysis chemistry. Upon cartridge insertion, the reader actuates the blister, initiates a timed transfer sequence, and then acquires endpoint fluorescence after a reaction interval of approximately fifteen minutes. The premium design reduces operator steps while preserving the same cartridge-reader product identity.

Example 5. A low-cost variant uses manual lysis in a small vial followed by transfer of an aliquot to the cartridge inlet. After sealing, the cartridge remains closed throughout incubation and readout. This embodiment demonstrates that the invention can be commercialized at a lower instrument-complexity tier without losing the fundamental value of the point-of-care molecular cartridge-reader architecture.

Example 6. A cartridge lot is encoded with calibration metadata readable by the reader. The processor applies lot-specific threshold adjustments and optical normalization factors before classifying a run. The resulting system improves consistency across manufactured lots and supports scalable deployment in multi-reader installations.

Example 7. A contamination-control embodiment includes a sealed waste chamber containing absorbent material and a unidirectional flow path separated from the read window. After the run, the cartridge can be removed and discarded without reopening any fluidic chamber. This embodiment is valuable in decentralized settings that require safe handling by operators with limited laboratory training.

Example 8. A smartphone or tablet provides the user interface for the reader. The reader performs heating and optical acquisition locally, while the external device displays operator instructions, timer status, and final triage category. The communications link may be wired or wireless and may also support export of a summarized run log.

Example 9. A benchtop clinic embodiment uses a richer display, additional service menus, and supervisory controls while preserving the same assay cartridge. This illustrates the commercial flexibility of a common consumable family paired with multiple instrument tiers.

Example 10. A kit includes cartridges, cervical swabs, lysis buffer, positive and negative control materials, and instructions for same-visit screening. The kit is supplied in a lot-coded package with shelf-life and quality markings that are machine-readable by the reader or by a supporting inventory tool.

Example 11. A service diagnostic routine uses a calibration article to verify the excitation source, detector gain, and heater performance of the reader. The routine records service data separately from patient-facing assay data, thereby supporting fleet maintenance without confusing clinical logs.

Example 12. A workflow embodiment is used in a mobile screening vehicle. The operator collects a cervical sample, performs a simplified transfer, and receives a triage result during the same patient encounter. The portable nature of the reader and sealed consumable format enable meaningful operation outside a central laboratory.

Example 13. A commercialization embodiment packages the cartridge-reader system as a licensable module for integration into an established women's health testing business. The buyer or licensee can adapt branding, user-interface language, and data-export settings while relying on the core disclosed cartridge architecture, optical readout, and triage logic.

Example 14. A reaction-design embodiment uses one region for HPV16, one region for HPV18, one region for grouped high-risk HPV, and one region for internal control. The grouped high-risk region provides clinically meaningful information without requiring the reader to identify every individual subtype, enabling a practical balance between clinical utility and compact instrument design.

Example 15. A manufacturing embodiment uses roll-to-roll processing for cover films and batch loading of dried reagents into molded base substrates. Optical-window inspection and seal-integrity testing are performed before pouching. This embodiment demonstrates a realistic path from development to scaled production.

Example 16. A quality-control embodiment stores confidence-related metadata associated with each run while presenting a simplified result to the ordinary operator. Supervisory personnel can review additional data when investigating borderline behavior, thereby improving supportability and reducing the burden of decentralized deployment.

Example 17. A premium software embodiment applies deterministic thresholding together with a secondary consistency check that examines expected relationships among control and test regions. The secondary check does not replace the molecular invention but instead suppresses issuance of questionable outputs that could otherwise erode field trust.

Example 18. A cartridge family uses the same docking footprint and optical window geometry across several assay menus. This allows a portfolio owner to commercialize related women's health assays while leveraging a common reader platform, a strategy that can materially enhance the transaction value of the disclosed core architecture.

Example 19. A distribution embodiment uses individually pouched cartridges shipped with humidity control and machine-readable expiration data. The reader can refuse to start a run when the cartridge is expired or when the lot code is not recognized, thereby supporting safer field deployment and improved quality control.

Example 20. A result-reporting embodiment provides one of the categories HPV 16/18 positive, other high-risk HPV positive, negative, or invalid, and additionally stores a run identifier, assay version, and lot identifier. This combination supports both clinical usability and traceability, which are important for licensing and product adoption.

Prophetic Example 20A. A clinic-deployable embodiment uses a rectangular hand-loadable cartridge having a principal reaction microchannel of about 100 micrometers width and about 50 micrometers depth, together with on-reader thermal control at about 37 degrees Celsius for about 15 minutes and direct optical interrogation through a read window, to support issuance of a categorized same-visit triage output.

Prophetic Example 20B. In another representative embodiment, the coordinated use of confined microfluidic reaction geometry, near-isothermal CRISPR timing, and direct optical readout in a closed cartridge-reader workflow reduces dependence on remote laboratory batching and extended post-processing, thereby supporting faster availability of actionable triage information.

### Validation, Performance, and Development Support

Validation work may be carried out with emphasis on analytical sensitivity. For example, limit-of-detection studies using representative target materials and dilution series can be used to identify operating windows that preserve clinically useful detection while remaining compatible with compact optics and low reagent volume. The present disclosure is intentionally drafted to accommodate such validation activity because a patent asset aimed at product transfer should support not merely laboratory curiosity, but a path toward reproducible commercial execution.

Validation work may be carried out with emphasis on specificity and cross-reactivity. For example, cross-reactivity testing against non-target nucleic acids, commensal flora, and non-target HPV classes can be used to refine guide selection and threshold rules. The present disclosure is intentionally drafted to accommodate such validation activity because a patent asset aimed at product transfer should support not merely laboratory curiosity, but a path toward reproducible commercial execution.

Validation work may be carried out with emphasis on repeatability. For example, repeat runs across multiple readers, lots, operators, and environmental conditions can be used to set acceptable coefficients of variation and lot-release criteria. The present disclosure is intentionally drafted to accommodate such validation activity because a patent asset aimed at product transfer should support not merely laboratory curiosity, but a path toward reproducible commercial execution.

Validation work may be carried out with emphasis on stability. For example, accelerated and real-time stability studies can be used to determine pouching strategy, desiccant needs, and reagent format selection. The present disclosure is intentionally drafted to accommodate such validation activity because a patent asset aimed at product transfer should support not merely laboratory curiosity, but a path toward reproducible commercial execution.

Validation work may be carried out with emphasis on usability. For example, formative and summative operator studies can be used to refine insertion cues, prompts, and training materials. The present disclosure is intentionally drafted to accommodate such validation activity because a patent asset aimed at product transfer should support not merely laboratory curiosity, but a path toward reproducible commercial execution.

Validation work may be carried out with emphasis on field robustness. For example, mobile-clinic and decentralized-use studies can be used to confirm that the cartridge-reader combination remains useful outside central-laboratory conditions. The present disclosure is intentionally drafted to accommodate such validation activity because a patent asset aimed at product transfer should support not merely laboratory curiosity, but a path toward reproducible commercial execution.

Validation work may be carried out with emphasis on invalid-rate management. For example, systematic review of control failures and borderline runs can be used to optimize invalid thresholds and repeat-test guidance. The present disclosure is intentionally drafted to accommodate such validation activity because a patent asset aimed at product transfer should support not merely laboratory curiosity, but a path toward reproducible commercial execution.

Validation work may be carried out with emphasis on manufacturing consistency. For example, process-capability review of channel geometry, optical window quality, seal integrity, and reagent loading can be used to tighten commercial release criteria. The present disclosure is intentionally drafted to accommodate such validation activity because a patent asset aimed at product transfer should support not merely laboratory curiosity, but a path toward reproducible commercial execution.

The specification also contemplates prophetic and developmental examples that guide optimization without implying that every possible embodiment has already been reduced to practice. Such drafting is appropriate for a commercial patent filing because it provides meaningful technical instruction while preserving flexibility for later engineering refinement and continuation practice.

A useful commercialization sequence may therefore involve bench feasibility, cartridge optimization, reader optimization, pilot usability, stability work, and scaled manufacturing support. The disclosure supports that sequence because the strongest patent-backed asset package often couples a well-drafted device nucleus with a clear validation roadmap.

### Advantages and Commercial Utility

The invention can provide rapid turnaround, subtype-aware molecular screening, compact optics, low reagent consumption, sealed waste handling, practical same-visit output, and modular commercialization pathways. Because the disclosure centers on a concrete cartridge-reader configuration rather than on an unfocused platform narrative, it is better positioned for examination, transfer, licensing, and integration into a real product line.

Rapid turnaround in the present architecture arises from a coordinated combination of confined microfluidic geometry, near-isothermal reaction control, and direct optical readout in a closed sample-to-answer path. Together these features can shorten diffusion distance, reduce reagent volume, support faster thermal equilibration, and avoid extended off-cartridge post-processing before triage output generation.

A transaction counterparty evaluating the present invention can identify a consumable article, an instrument article, defined assay timing, defined optical behavior, defined output categories, and kit-level deployment concepts. This is materially more useful than a broad story lacking a buildable core. The disclosed structure therefore supports not only potential allowance prospects but also higher practical value in assignment, licensing, or structured asset-package discussions.

The invention is further useful as a portfolio anchor because the current application can support additional claim families directed to cartridge architecture, reader structure, controls, kit features, manufacturing, and product variants. A coherent core application with follow-on claiming potential is generally more valuable than a diffuse first filing that is difficult to examine or difficult to map to a product.

### Alternative Embodiments and Variations

Alternative embodiments may use different channel counts, excitation wavelengths, detector types, reporter formats, reagent-storage formats, materials, docking features, or user interfaces, provided that the integrated point-of-care cartridge-reader concept is preserved. Such variations do not depart from the invention merely because they optimize cost, field durability, or customer-specific requirements.

Alternative embodiments may substitute partial automation for full automation, or vice versa. They may use a smartphone as the display layer, a benchtop housing, or a service-connected fleet model. They may add calibration accessories, supervisory menus, or region-specific assay content. The disclosure is intended to support these commercially sensible evolutions without abandoning the focused identity of the presently claimed invention.

The foregoing description is not intended to limit the invention to the precise forms disclosed. Modifications and variations are possible in light of the above teaching. The embodiments were selected and described to explain the principles of the invention, its practical application, and its commercial relevance, thereby enabling others skilled in the art to utilize the invention in various embodiments and with various modifications suited to particular contemplated uses.

The invention is defined by the claims that follow and equivalents thereof.

### Guide-design Principles and Target Selection

Guide design may be based on conserved sequence motifs, subtype-discriminating motifs, mismatch tolerance analysis, and empirical signal behavior. In some embodiments, separate design tracks are maintained for HPV16, HPV18, and a grouped high-risk class. In some embodiments, design filters consider local secondary structure, off-target risk against related HPV classes, expected sample abundance, and compatibility with rapid endpoint acquisition in a compact reader.

A preferred commercialization pathway uses a stable first-generation assay menu and preserves later flexibility to refresh or replace guide sets without changing the fundamental cartridge-reader footprint. This is valuable because target evolution, market requirements, or competitive positioning may justify chemistry updates while the commercial platform remains physically consistent. The specification therefore supports product-line continuity together with assay-content refinement.

In some embodiments, the grouped high-risk class is defined by a rationally selected subset of subtypes that align with the intended screening environment, reimbursement environment, or partner preference. In some embodiments, the grouped class is intentionally broader and sacrifices subtype granularity for speed and simplicity. In still other embodiments, grouped and subtype-specific regions are both present, thereby allowing a compact but clinically informative result set.

Design rules may also incorporate control over guide redundancy. For example, multiple guide elements may be placed in a single region to increase robustness while still preserving a single optical reporting region. Alternatively, one guide set may be preferred to simplify reagent composition. Such alternatives are relevant to both patent support and manufacturing strategy because they influence cost, yield, and performance.

The disclosure deliberately captures guide-design logic at a level that is technically meaningful without locking the patent asset to a single laboratory recipe. That balance helps preserve future optimization freedom while still strengthening written-description support for a real-world product family.

### Signal Processing and Decision Architecture

Signal processing may include dark-frame capture, background subtraction, normalization to a reference or internal-control region, time-window smoothing, endpoint comparison, trend analysis, or hybrid processing. In some embodiments, the processor computes one or more derived metrics before comparing the result to stored thresholds. In some embodiments, the processing path differs for genotype-specific regions and grouped-target regions.

The processor may also evaluate relationships among multiple regions rather than reading each region in isolation. For example, a grouped high-risk region may be interpreted differently depending on whether a subtype-specific region is strongly positive. Such logic can be implemented using deterministic rule tables that preserve claim clarity and reduce unnecessary dependence on abstract software language.

Decision architecture may further include invalid suppression, repeat recommendation, or quality escalation outputs. For example, where control behavior is borderline, the system may prefer a repeat-test classification rather than a definitive negative classification. These outcome-management behaviors are commercially meaningful because they reduce field risk and support adoption in settings where confirmatory laboratory staff are not immediately present.

In some embodiments, the processor stores raw or semi-processed intensity values for later review. In some embodiments, only summarized values are retained to reduce storage burden. In still other embodiments, a premium software tier retains enhanced diagnostics while a basic tier retains only clinical categories. The disclosure supports these variants because different business models may value transparency, storage cost, and service complexity differently.

The integration of signal processing with physical reader and cartridge design is important. Optical geometry, channel dimensions, and control chemistry all influence the processing rules that are practical. The present application therefore describes the decision architecture as part of the same device story rather than as a standalone data-processing concept.

### Environmental Robustness and Field Conditions

The cartridge-reader system may be designed for operation across a range of ambient temperatures, humidity conditions, transport exposures, and power environments. In some embodiments, the reader performs ambient compensation or warm-up adaptation. In some embodiments, the cartridge pouching strategy is selected to preserve reagent quality despite intermittent exposure during shipping or site storage.

Field deployment may require tolerance to vibration, dust, uneven power quality, and operator turnover. Accordingly, ruggedized embodiments may include shock-tolerant optical alignment, sealed buttons or touch surfaces, fanless thermal management, and self-check routines that run at power-on or cartridge insertion. Such details strengthen the patent asset because they connect the invention to real deployment, not merely bench aspiration.

A mobile-screening embodiment may prioritize low power draw, battery-backed operation, simple consumable handling, and clear status messaging. A clinic-fixed embodiment may prioritize serviceability, throughput, supervisory controls, and richer audit logging. The specification supports both classes because the commercial value of the patent rises when a common core can support multiple deployment tiers.

Environmental robustness also includes biological robustness. In some embodiments, the system accommodates routine variation in specimen burden, preservative carryover, or mucus content through a combination of lysis strategy, control logic, and fluidic tolerance. Where that tolerance is exceeded, invalid suppression provides a safer path than forced over-interpretation.

Because field suitability often influences acquisition and licensing interest, the present disclosure repeatedly ties technical features such as closed handling, temperature control, and control verification back to real deployment conditions. This alignment between engineering and deployment is part of what makes the invention commercially weighty.

### Reader Serviceability and Fleet Management

Serviceability may be designed into the reader at the hardware, firmware, and workflow levels. Modular subassemblies, diagnostic menus, calibration cartridges, replaceable covers, and software-readable error logs can all reduce field service burden. These considerations are not peripheral: they can materially influence the willingness of a commercial partner to adopt or license a platform.

In some embodiments, a service technician can verify heater uniformity, detector response, and optical cleanliness without opening the assay chemistry portion of the system. In some embodiments, self-test routines run automatically and log maintenance-related events separately from patient-facing assay events. Such separation preserves record clarity while enabling fleet support.

Fleet management may involve assigning reader identifiers, tracking firmware revisions, monitoring lot compatibility, and reviewing invalid or error rates across an installed base. The disclosed logging and calibration concepts support such practices even when the primary claimed invention remains device-centered and patient-facing in its ordinary use.

A commercial owner may also use serviceability features to segment offerings. For example, a basic single-site product may use a minimal diagnostic stack, whereas a fleet-managed enterprise version may use richer service telemetry. The same patent nucleus can support both, which in turn increases portfolio leverage.

By addressing serviceability in the specification, the application supports a more sophisticated asset story: not only can the platform generate a result, it can also be maintained, audited, and scaled. That is exactly the kind of practical completeness that can increase transaction credibility.

### Additional Cartridge Variants and Consumable Features

The cartridge may incorporate peel seals, pierceable membranes, compression zones, sacrificial break regions, integrated desiccant cavities, or tamper-evident features. These consumable details may appear modest, but they often influence product differentiation, manufacturability, and leakage performance. Their inclusion in the specification broadens support for downstream claim refinement.

Some variants may use an optical index region that confirms correct cartridge type before a run begins. Others may use molded fiducials to improve optical registration or detect warped consumables. Others may use pressure-equalization vents or selective vent membranes to improve fill consistency. Each such feature can matter if competitive products converge on a similar high-level workflow but differ in execution details.

Cartridge families may also vary in how they partition control materials. One family may use a dedicated internal-control region. Another may use multiple control regions addressing chemistry and transfer separately. Another may embed passive references in the optical window while using a biological control elsewhere. The present specification is intentionally rich enough to support these variations.

A premium consumable may include on-board reagent release triggered by reader mechanics. A value consumable may instead expect pre-lysis and manual loading. Both variants remain within the same commercial logic because they share a cartridge-reader relationship, defined reaction environment, and categorized triage output.

The present disclosure therefore supports a commercially realistic consumable roadmap extending from low-complexity entry products to premium integrated consumables. That roadmap can be valuable in prosecution and even more valuable in licensing negotiations.

### Program Integration and Clinical Utility

Clinical utility is improved when the assay result is available in time to affect immediate counseling, scheduling, or referral planning. The system is therefore especially suited to same-visit women's health encounters in which patient follow-up risk would otherwise increase if results were delayed. This practical advantage gives the invention significance beyond a purely academic assay demonstration.

In some embodiments, the triage categories are mapped to local practice rules or partner-defined workflows. In some embodiments, a site may choose to display only category labels. In other embodiments, a site may display associated procedural prompts. The reader can accommodate both while preserving the same physical and assay architecture.

A healthcare partner considering license or integration often evaluates whether a platform can be adapted to different care settings without undermining its core value proposition. By disclosing multiple program contexts while maintaining a narrow primary embodiment, the present application supports that adaptability without drifting into diffuse abstraction.

The system also supports more efficient use of clinician time. If a patient encounter can end with a categorized output rather than with an open-ended delay, counseling and routing can be more deliberate. Such workflow benefits are not merely commercial rhetoric; they are a direct consequence of the disclosed combination of compact reader, sealed cartridge, controlled reaction, and integrated triage logic.

Because real adoption depends on usefulness at the point of care, the specification repeatedly frames the invention as a practical module that can be built, sold, tested, and used. This emphasis is appropriate for a patent intended to function as both a prosecution asset and a licensing asset.

### Extended Example Set

Example 21. An embodiment uses a cartridge read window treated to reduce autofluorescence, thereby improving discrimination of the approximately 605-nanometer signal in a compact optical path. This optical refinement cooperates with the reader filter set and the selected reporter chemistry to improve field robustness without materially increasing operator burden.

Example 22. An embodiment uses a lot-coded pouch and reader verification routine that blocks a run if the cartridge lot is incompatible with the installed assay definition. This improves deployment control and helps a fleet owner manage upgrades or chemistry refreshes across multiple sites.

Example 23. An embodiment uses a dual-stage warm-up sequence in which the reader first stabilizes the bay and then starts the assay timing interval only after the reaction zone enters the intended range. This supports more consistent same-visit performance across varying ambient conditions.

Example 24. An embodiment stores summarized raw-intensity values together with the final category, allowing later review during quality audits while still presenting a simple patient-facing output to the ordinary operator. This is valuable in commercial rollouts that require traceability but not constant data overload.

Example 25. An embodiment uses different optical integration times for the control region and the grouped-target region, thereby improving dynamic range while preserving a compact single-detector architecture. This example illustrates how the processor and optics can be co-designed around cartridge layout.

Example 26. An embodiment uses a sealed accessory vial pre-loaded with lysis chemistry and a transfer geometry keyed to the cartridge inlet. The keyed transfer path reduces the likelihood of user error and helps maintain closed handling through most of the workflow.

Example 27. An embodiment adds a duplicate control region to improve discrimination between chemistry failure and sample-quality failure. This can be useful in higher-end product variants or regulated deployments where more granular invalid reasoning is commercially worthwhile.

Example 28. An embodiment packages the reader with service prompts and preventive-maintenance thresholds so that a fleet operator can proactively replace or clean parts before field failures accumulate. Such features improve the transaction profile of the platform by showing operational maturity.

Example 29. An embodiment provides a cartridge body with molded registration features that align both the thermal interface and the optical path. This combined registration strategy can reduce run-to-run variation and make the device architecture more defensible as a concrete engineered product.

Example 30. An embodiment configures a grouped high-risk region to favor practical triage speed over maximal subtype granularity. This illustrates a commercially rational design choice and supports claims directed to clinically useful grouped outputs rather than to exhaustive laboratory typing.

Example 31. An embodiment uses a cartridge read window treated to reduce autofluorescence, thereby improving discrimination of the approximately 605-nanometer signal in a compact optical path. This optical refinement cooperates with the reader filter set and the selected reporter chemistry to improve field robustness without materially increasing operator burden.

Example 32. An embodiment uses a lot-coded pouch and reader verification routine that blocks a run if the cartridge lot is incompatible with the installed assay definition. This improves deployment control and helps a fleet owner manage upgrades or chemistry refreshes across multiple sites.

Example 33. An embodiment uses a dual-stage warm-up sequence in which the reader first stabilizes the bay and then starts the assay timing interval only after the reaction zone enters the intended range. This supports more consistent same-visit performance across varying ambient conditions.

Example 34. An embodiment stores summarized raw-intensity values together with the final category, allowing later review during quality audits while still presenting a simple patient-facing output to the ordinary operator. This is valuable in commercial rollouts that require traceability but not constant data overload.

Example 35. An embodiment uses different optical integration times for the control region and the grouped-target region, thereby improving dynamic range while preserving a compact single-detector architecture. This example illustrates how the processor and optics can be co-designed around cartridge layout.

Example 36. An embodiment uses a sealed accessory vial pre-loaded with lysis chemistry and a transfer geometry keyed to the cartridge inlet. The keyed transfer path reduces the likelihood of user error and helps maintain closed handling through most of the workflow.

Example 37. An embodiment adds a duplicate control region to improve discrimination between chemistry failure and sample-quality failure. This can be useful in higher-end product variants or regulated deployments where more granular invalid reasoning is commercially worthwhile.

Example 38. An embodiment packages the reader with service prompts and preventive-maintenance thresholds so that a fleet operator can proactively replace or clean parts before field failures accumulate. Such features improve the transaction profile of the platform by showing operational maturity.

Example 39. An embodiment provides a cartridge body with molded registration features that align both the thermal interface and the optical path. This combined registration strategy can reduce run-to-run variation and make the device architecture more defensible as a concrete engineered product.

Example 40. An embodiment configures a grouped high-risk region to favor practical triage speed over maximal subtype granularity. This illustrates a commercially rational design choice and supports claims directed to clinically useful grouped outputs rather than to exhaustive laboratory typing.

Example 41. An embodiment uses a cartridge read window treated to reduce autofluorescence, thereby improving discrimination of the approximately 605-nanometer signal in a compact optical path. This optical refinement cooperates with the reader filter set and the selected reporter chemistry to improve field robustness without materially increasing operator burden.

Example 42. An embodiment uses a lot-coded pouch and reader verification routine that blocks a run if the cartridge lot is incompatible with the installed assay definition. This improves deployment control and helps a fleet owner manage upgrades or chemistry refreshes across multiple sites.

Example 43. An embodiment uses a dual-stage warm-up sequence in which the reader first stabilizes the bay and then starts the assay timing interval only after the reaction zone enters the intended range. This supports more consistent same-visit performance across varying ambient conditions.

Example 44. An embodiment stores summarized raw-intensity values together with the final category, allowing later review during quality audits while still presenting a simple patient-facing output to the ordinary operator. This is valuable in commercial rollouts that require traceability but not constant data overload.

Example 45. An embodiment uses different optical integration times for the control region and the grouped-target region, thereby improving dynamic range while preserving a compact single-detector architecture. This example illustrates how the processor and optics can be co-designed around cartridge layout.

Example 46. An embodiment uses a sealed accessory vial pre-loaded with lysis chemistry and a transfer geometry keyed to the cartridge inlet. The keyed transfer path reduces the likelihood of user error and helps maintain closed handling through most of the workflow.

Example 47. An embodiment adds a duplicate control region to improve discrimination between chemistry failure and sample-quality failure. This can be useful in higher-end product variants or regulated deployments where more granular invalid reasoning is commercially worthwhile.

Example 48. An embodiment packages the reader with service prompts and preventive-maintenance thresholds so that a fleet operator can proactively replace or clean parts before field failures accumulate. Such features improve the transaction profile of the platform by showing operational maturity.

Example 49. An embodiment provides a cartridge body with molded registration features that align both the thermal interface and the optical path. This combined registration strategy can reduce run-to-run variation and make the device architecture more defensible as a concrete engineered product.

Example 50. An embodiment configures a grouped high-risk region to favor practical triage speed over maximal subtype granularity. This illustrates a commercially rational design choice and supports claims directed to clinically useful grouped outputs rather than to exhaustive laboratory typing.

Example 51. An embodiment uses a cartridge read window treated to reduce autofluorescence, thereby improving discrimination of the approximately 605-nanometer signal in a compact optical path. This optical refinement cooperates with the reader filter set and the selected reporter chemistry to improve field robustness without materially increasing operator burden.

Example 52. An embodiment uses a lot-coded pouch and reader verification routine that blocks a run if the cartridge lot is incompatible with the installed assay definition. This improves deployment control and helps a fleet owner manage upgrades or chemistry refreshes across multiple sites.

Example 53. An embodiment uses a dual-stage warm-up sequence in which the reader first stabilizes the bay and then starts the assay timing interval only after the reaction zone enters the intended range. This supports more consistent same-visit performance across varying ambient conditions.

Example 54. An embodiment stores summarized raw-intensity values together with the final category, allowing later review during quality audits while still presenting a simple patient-facing output to the ordinary operator. This is valuable in commercial rollouts that require traceability but not constant data overload.

Example 55. An embodiment uses different optical integration times for the control region and the grouped-target region, thereby improving dynamic range while preserving a compact single-detector architecture. This example illustrates how the processor and optics can be co-designed around cartridge layout.

### Further Commercial and Portfolio Considerations

The present specification is structured to support a prosecution strategy in which the current claims remain centered on a concrete point-of-care cartridge-reader product while future continuation or divisional practice can selectively emphasize cartridge structure, reader mechanics, control logic, kit format, or manufacturing details. That layered approach can improve portfolio resilience and make the resulting asset package easier to explain to prospective buyers or licensees.

Equally important, the disclosure avoids depending for its identity on abstract software, reimbursement rules, or generalized data-routing concepts. Instead, it presents a tangible molecular device system that can be valued, prototyped, diligence-reviewed, and integrated into a product family. This practical orientation is a major reason why the application can serve as a strong market-facing asset if prosecution is handled carefully.

### Expanded Embodiments, Use Cases, and Engineering Detail

The cartridge may include a staged fill architecture in which an initial portion of the specimen wets a priming segment before entering a reaction segment. This can reduce bubble formation, improve reproducibility of reaction-volume distribution, and provide a more stable optical read region. Such details are especially relevant when the platform is scaled from prototype use to high-volume manufacturing.

In some embodiments, the optical read region is intentionally separated from a more chemically aggressive lysis region by a defined transfer path. This spatial separation can reduce optical fouling and preserve the long-term accuracy of calibration assumptions. It also gives the cartridge architecture a clearer functional partitioning that may later support claim refinement.

An embodiment may use a cartridge label or molded indicia indicating the intended panel version so that a clinic can visually distinguish consumables while the reader still performs machine verification. The combined human-plus-machine identification strategy is useful where multiple assay menus share a common reader footprint.

In some embodiments, the reader enclosure includes a shading geometry or bay cover that reduces the effect of room lighting on optical acquisition. This may permit operation in a broader range of clinic and field environments without materially increasing instrument complexity.

An embodiment may include an operator-guidance sequence that displays only the next required action, thereby reducing training burden. For example, the interface may progress through sample loaded, cartridge inserted, run active, and result ready states without exposing unnecessary technical details to the ordinary user.

In some embodiments, the cartridge includes a sacrificial dead-volume segment that captures the first-arriving portion of the transfer stream before the more representative portion reaches the read-sensitive reaction channel. Such a design can improve consistency where sample-conditioning variability is expected.

In some embodiments, the assay is packaged as a clinical-screening product module intended for distribution through third-party women's health networks. In such a module, branding, packaging, and software presentation may vary by partner while the core consumable-and-reader architecture remains substantively unchanged.

The reader may be configured to refuse initiation of a run until a lid, bay closure, or insertion sensor confirms correct cartridge seating. This helps preserve optical geometry, reduces user error, and supports safer and more reproducible field operation.

In some embodiments, the cartridge and reader are jointly configured to provide a predictable thermal mass and optical path, thereby minimizing the amount of complicated compensation required in firmware. This kind of co-design can materially improve manufacturability and service behavior.

An embodiment may use color-coded accessory components associated with sample collection, lysis, and cartridge loading. Such ergonomic features are commercially useful in decentralized operation and help demonstrate that the invention has been considered as a real workflow rather than an isolated reaction diagram.

In some embodiments, the processor logs whether a result was issued normally, suppressed as invalid, or flagged for repeat recommendation. Such event categorization can be valuable for quality trending over time and for comparing reader performance across an installed base.

The disclosure also contemplates that a commercial owner may package the platform with training materials, workflow prompts, or implementation support adapted to the intended customer type. Because the core technical platform is concrete, these commercialization layers can be added without undermining the patent nucleus.

In some embodiments, the cartridge includes a physical feature that discourages reuse, such as a latch fracture region, irreversible seal element, or use-indicator. Single-use integrity can matter clinically and commercially, and therefore supports additional continuation possibilities if needed.

The optical detection subsystem may use endpoint reading, multiple endpoint snapshots, or a short temporal acquisition window sufficient to improve confidence while still fitting a same-visit workflow. The specification supports each variant because the central invention lies in the integrated device pathway rather than one rigid timing script.

In some embodiments, the system is optimized for simple deployment with minimal calibration burden at the customer site. In others, a partner-oriented version may include more granular calibration and service features to support enterprise integration. Such scalable architecture can increase transaction value.

The disclosed platform is also suitable for staged portfolio development. A first filing can emphasize the core point-of-care product combination, while later filings can selectively emphasize reader subassemblies, cartridge geometries, kit packaging, or control strategies. The present specification is drafted with that commercial reality in mind.

An embodiment may employ a protected optical read pocket that isolates the measurement region from operator touch, ambient splashes, and visible contamination. This contributes both to measurement integrity and to a more professional field-ready product appearance.

In some embodiments, the system is configured to support internal lot qualification by allowing bench runs with defined controls before distribution. These qualification pathways are commercially important because they affect partner confidence and can be relevant during diligence or transfer discussions.

The cartridge may include structures that slow, meter, or distribute fluid without active pumping. Such passive fluid management can materially reduce instrument complexity and cost, which is often crucial in decentralized women's health screening products.

Conversely, higher-end embodiments may use reader-actuated mechanics to gain tighter timing control or reduce user steps. Both low-complexity and high-complexity variants remain within the scope of a coherent cartridge-reader invention and can be monetized differently in the market.

The specification is written so that a party evaluating the asset can identify not only an assay concept but also a productizable pathway involving materials, consumables, instrumentation, control logic, packaging, and deployment. That integrated completeness is a major component of strategic value.

The system can also support pilot-market deployment strategies in which small quantities of consumables are supplied to selected clinics for workflow validation before a broader rollout. The practical orientation of the present disclosure makes such staged commercialization easier to explain and support.

In some embodiments, an operator account, site code, or reader identifier is associated with the run record. This is helpful in networks where fleet-level quality monitoring or inventory control matters. It also reinforces the platform's readiness for commercial operation.

An embodiment may use a cartridge geometry optimized for nesting and transport efficiency. Seemingly simple packaging geometry can influence shipping cost and field stocking behavior, which are highly relevant to market adoption and therefore to portfolio value.

Where appropriate, reader software may include region-or partner-specific result text while retaining the same core category logic. This allows localization and commercialization flexibility without changing the physical cartridge and optical system that anchor the patent story.

The present disclosure also supports the idea that a platform owner may license the consumable and the reader together, or may license only one of those components under a structured arrangement. A patent asset with support for both views can be more valuable in negotiation.

The cartridge may be manufactured with optical fiducials enabling automated inspection during production. This can improve yield and reduce the risk of shipping consumables that would later fail in the field. Such design-for-manufacture detail is part of what can elevate the practical significance of the patent asset.

An embodiment may incorporate anti-tamper coding or controlled firmware recognition to reduce unauthorized use of incompatible cartridges. While such business arrangements are optional, the specification supports them because they may become relevant in higher-value deployments.

The reader may display a countdown synchronized to the actual reaction-control state rather than to a crude fixed timer. This can improve user confidence and reduce premature handling of the cartridge before the assay is ready for readout.

In some embodiments, reader memory stores reference values used to detect abnormal instrument drift. This can permit earlier service intervention and preserve the credibility of a decentralized installed base.

The specification also supports bench-scale research variants in which additional hidden diagnostic data are collected, even though the ordinary commercial version may present only a concise categorized result. This allows the same platform family to serve both development and deployment needs.

The disclosed architecture may be embodied as a product sold directly, licensed to a diagnostic company, transferred as part of a larger women's health asset package, or used as an anchor for a continuation family. The application is drafted to support all of these pathways without departing from its focused technical identity.

The strength of the present invention lies not in any one buzzword, but in the disciplined combination of sample handling, microfluidic structure, reaction control, optical readout, and triage packaging. This combination is what gives the patent both prosecutable shape and real commercial meaning.

In some embodiments, the platform is paired with customer training, consumable supply, and service support as a bundled offering. The present disclosure is suitable for that kind of bundled commercialization because it covers the critical elements that such a business model would rely on.

An embodiment may provide supervisory access to run-history analytics such as invalid rate by lot, site, or reader. Such analytics can help a commercial operator identify user-training issues, shipping issues, or maintenance needs, thereby reducing field costs.

The platform may also be offered through regional private-label arrangements in which partner-specific packaging is used. Because the core technical module remains the same, the patent can still support strong transaction positioning even across different commercial wrappers.

The specification is intentionally rich in optional but practical engineering features so that later prosecution can adapt to the prior-art landscape without losing access to meaningful fallback positions. This is a substantial part of what makes the application suitable as a high-value asset foundation.

Where a prospective licensee wishes to emphasize consumable economics, the cartridge-related passages of the present specification provide strong grounding. Where the licensee wishes to emphasize instrument differentiation, the reader-related passages provide similar grounding. This dual attractiveness can enhance negotiation leverage.

An embodiment may include controlled disposal instructions, waste containment accessories, or post-use packaging to support safer clinical handling. Such features matter in real deployment and are appropriately reflected in a patent specification intended to support actual market use.

Ultimately, the disclosed system is designed to be more than a scientific thought experiment. It is meant to function as a licensable, buildable, and scalable women's health molecular testing module whose value can be explained to examiners, engineers, investors, and commercial partners alike.

### Further Embodiments and Claim-Support Variants

In some embodiments, the cartridge defines a localized reaction cavity downstream of a narrower transfer channel so that optical readout occurs in a region optimized for measurement while transfer occurs in a region optimized for sequencing. This partitioning supports a refined product geometry without changing the overall cartridge-reader logic.

In some embodiments, the cartridge is manufactured with region-specific surface treatment so that one area emphasizes rapid wetting and another emphasizes signal stability. Such selective treatment can be useful when reagent rehydration and optical measurement benefit from different local conditions.

In some embodiments, a site-deployable starter package includes a reader, a first lot of cartridges, quality-control materials, quick-reference guides, and maintenance prompts. Packaging a platform in this way can accelerate adoption and improve transaction readiness.

In some embodiments, the platform is introduced first in centralized outpatient clinics and later extended into decentralized screening. The specification supports that commercial sequence because the same cartridge-reader core can satisfy both early controlled rollout and later scaled deployment.

In some embodiments, a cartridge includes a peelable or pierceable access structure sealed after filling during manufacture, while the user-facing sample entry remains separate. This can simplify reagent loading and increase manufacturing flexibility.

In some embodiments, the system uses an endpoint reading supplemented by one or more pre-read checks that confirm optical baseline and thermal readiness. This can reduce false calls arising from premature acquisition.

In some embodiments, control logic distinguishes between chemistry failure, fluid-transfer failure, and reader fault based on the pattern of signals across multiple regions and self-check outcomes. More informative invalid handling can be valuable in service-heavy deployments.

In some embodiments, the reader stores cartridge compatibility tables that permit the same instrument to support successive generations of consumables. This can preserve installed-base value and improve commercial attractiveness to partners who care about product-roadmap continuity.

In some embodiments, an enterprise-oriented product version includes site-level supervisory access for inventory and run-history review. Even though such functions are not needed in every deployment, the underlying technical module benefits from being described as adaptable to them.

In some embodiments, the cartridge includes a geometry that physically spaces used-sample material from the optical read window to reduce residual fouling or post-run visual contamination. This contributes both to measurement reliability and to user confidence.

In some embodiments, the assay menu is intentionally limited to a clinically actionable subset of high-risk categories so that the instrument can remain compact and easy to use. Such disciplined product design can make the resulting patent asset more meaningful than an undisciplined attempt to claim every possible marker at once.

In some embodiments, the reader communicates a final category only after recording a complete audit entry. This sequencing can be useful in regulated or quality-managed environments where record completeness is important.

In some embodiments, the cartridge pouch includes external indicators showing storage condition, moisture exposure, or seal integrity. These features may also be machine-readable to further reduce field error.

In some embodiments, the reader can display step-specific warnings if a cartridge is inserted before sample loading or if the wrong cartridge family is detected. Such usability features support decentralized deployment and may become useful fallback claim material.

In some embodiments, the consumable is designed around low dead volume to preserve reagent economy. This can help make the platform more attractive in cost-sensitive markets.

In some embodiments, the reader uses an optical architecture selected to minimize moving parts. Reduced mechanical complexity can improve durability, serviceability, and total cost of ownership, each of which can contribute to transaction value.

In some embodiments, a partner-specific version of the product uses modified packaging and software language while retaining the same assay core. This reflects a realistic private-label or co-branded commercialization path.

In some embodiments, one or more regions are interrogated in a sequence that prioritizes control validation before test-region interpretation. Such sequencing can improve reader efficiency and reduce the chance of unnecessary downstream computation on clearly invalid runs.

In some embodiments, the system may be configured for local storage-only operation where network access is limited. In other embodiments, the same system may support export to external records. This flexibility improves deployment breadth without altering the primary invention.

In some embodiments, the cartridge includes molded strain-relief or reinforcement features near the inlet or docking edge. These can improve robustness during handling, shipping, or insertion.

In some embodiments, a supervisory tool is used to compare invalid rates across sites and readers. Such analysis can reveal shipping, operator-training, or maintenance issues, increasing the real-world value of the platform.

In some embodiments, the platform owner chooses to license only the cartridge family while supplying a reference reader design to a manufacturing partner. In other embodiments, the reader and consumables are licensed together. The specification supports both commercialization pathways.

In some embodiments, the kit includes replacement accessories or small field-service items such as cleaning swabs, alignment cards, or charging accessories. These features reinforce the practical, market-oriented character of the disclosed system.

In some embodiments, the reader may support multilingual user interfaces or region-specific result text without changing the underlying assay control logic. This increases international deployment flexibility.

In some embodiments, the cartridge is optimized for rapid pouch opening and direct loading in high-throughput clinic sessions. In other embodiments, it is optimized for rugged shipping and low-frequency field use. Both are supported.

In some embodiments, the system is used as a bridge between collection and referral decisions in settings where central laboratory access is delayed or inconsistent. The tangible utility of that bridge is one reason the invention can be commercially meaningful.

In some embodiments, the reader bay is shaped to discourage accidental touch of the optical window area during insertion. Such detail may appear minor, but it can materially influence long-term usability and maintenance behavior.

In some embodiments, a cartridge lot is released only after combined dimensional, reagent, and optical checks, thereby supporting stronger consistency claims in the marketplace. The patent specification benefits from reflecting such realistic quality practices.

In some embodiments, the platform is marketed as a core women's health molecular module capable of supporting multiple downstream business arrangements. The specification is intentionally written to make that commercial narrative credible without sacrificing technical focus.

In some embodiments, the same basic reader hardware supports software-locked assay menus activated according to customer needs. This can create flexible product segmentation while preserving the same mechanical and optical base.

In some embodiments, the user interface intentionally withholds raw numeric detail from ordinary users while making it available to qualified supervisors or service personnel. This can simplify operation without eliminating support capability.

In some embodiments, the system is integrated into a clinic workflow where specimen collection, loading, run initiation, counseling, and referral scheduling can all occur within one visit window. This workflow value is a direct outcome of the disclosed engineering choices.

In some embodiments, the patent asset is packaged together with validation plans, manufacturing notes, and commercial positioning materials. While those materials are outside the formal claim scope, the richness of the present specification makes that packaging more credible and valuable.

In some embodiments, the cartridge uses geometry and materials chosen to minimize warping under normal storage and operating temperatures. Optical repeatability and seal behavior can benefit from such design decisions.

In some embodiments, the reader includes an easily cleanable bay surface or removable insert to simplify maintenance between runs. Maintenance-friendly hardware can materially improve field performance.

In some embodiments, the platform is offered into regional or public-health tenders where the combination of same-visit utility, low operator burden, and consumable control is especially attractive. The invention is well suited to such structured deployment opportunities.

In some embodiments, a premium product version includes richer lot-traceability and diagnostics because those features can justify higher price and deeper service engagement. The specification supports that higher-value tier.

In some embodiments, the system is intentionally designed so that the most commercially distinctive features are tied to tangible hardware, chemistry, and workflow interactions rather than abstract business rules. This improves both patent posture and transaction clarity.

In some embodiments, the reader can log unsuccessful starts, expired-cartridge events, or user-cancel events. Such data may improve quality management and supply planning over time.

In some embodiments, a site can maintain mixed inventory of different cartridge families while using reader recognition and operator prompts to minimize mismatch. This is commercially useful when a platform owner expands menus over time.

In some embodiments, the consumable package includes a compact instruction card aligned to the actual sequence expected by the reader. The closer the packaging aligns with the device workflow, the lower the field-training burden is likely to be.

In some embodiments, the cartridge may be configured for automated optical inspection after manufacture so that contaminated or damaged windows are screened out before shipment. This reinforces the productizable nature of the invention.

In some embodiments, the platform may serve as an anchor for geographic licensing where one partner manufactures consumables and another distributes readers. The patent is drafted so that both sides of such a split can still see direct relevance.

In some embodiments, the assay timing window can be tuned around the same hardware architecture to support later chemistry improvements. This protects the long-term value of the installed base and can support more durable licensing economics.

In some embodiments, the system is attractive not because it tries to dominate all oncology workflows, but because it solves a focused and important women's health testing problem with enough technical specificity to be buildable and claimable. That disciplined focus is itself part of the asset's strength.

In some embodiments, a licensor may present the cartridge as the recurring-value consumable and the reader as the enabling installed instrument. The present specification supports that familiar commercial model because it discloses both sides of the architecture in practical detail.

In some embodiments, the current application acts as the core patent nucleus while later filings refine complementary themes. The richness of the present specification is intended to facilitate that portfolio architecture.

In some embodiments, the product may be used to reduce the operational gap between specimen collection and a categorized molecular result in settings where delayed laboratory pathways reduce follow-through. This practical impact is one reason why the invention can be truly useful.

In some embodiments, the platform's value in a market package is increased by the fact that the core technical story is coherent, the claims can be staged, and the commercial deployment logic is plausible. The present disclosure is drafted with all three considerations in view.

In some embodiments, the result pathway is intentionally narrow and disciplined so that the strongest claims can stay close to the tangible product. Broader ecosystem narratives, if desired, can be layered later without destabilizing the core application.

In some embodiments, the system offers a combination of actionable women's health relevance, engineering specificity, consumable economics, and upgradeable platform logic. That combination is exactly the sort of profile that can support licensing or transfer at a higher valuation than a diffuse or purely conceptual filing.

### Additional Detailed Support for Portfolio and Product Variants

The same core point-of-care architecture can support different commercialization tempos. A party may first prosecute and position the narrowest, strongest cartridge-reader claims, and only later activate broader product-family themes through continuation practice. The present specification is written to make that staged path believable and technically supported.

Because the invention is centered on a tangible product nucleus, it is well suited for diligence review. A counterparty can inspect the cartridge, understand the fluidic path, evaluate the optics and thermal design, and appreciate how the triage output is generated. This clarity can materially improve assignment or license conversations.

The specification also gives substantial attention to fallback detail. If prior art narrows one design pathway, other supported pathways remain available, including variants in materials, controls, reagent packaging, optical arrangement, reader service features, and workflow design. This is an important ingredient of a durable asset package.

In some embodiments, a partner may place special value on the recurring consumable economics of the cartridge. In other embodiments, the partner may focus more on the reader installation base. The present disclosure supports both views, which can expand the range of commercially interested counterparties.

An invention intended for real market use benefits from being able to accommodate both low-cost and premium product tiers. The current application expressly supports that flexibility through alternative embodiments that vary automation, user interface richness, calibration sophistication, and service infrastructure while preserving the same core molecular product logic.

The same disciplined architecture can also support geographic scaling. For example, an initial deployment may occur in controlled clinic settings, while later deployments may extend to mobile or decentralized screening. A coherent patent nucleus that anticipates these variations can be more valuable than one tied to a single narrow distribution assumption.

In some embodiments, the assay cartridge is supplied under subscription, standing-order, or consumable replenishment models. While such business structures are not themselves the patentable core, the fact that the technical platform supports them contributes to market packaging value.

Because the present filing focuses on a concrete women's health molecular module, it can operate as a portfolio anchor even if adjacent ecosystem concepts are pursued elsewhere. This separation of core module and broader ecosystem strategy can improve patent hygiene and preserve stronger examination posture for the main application.

In some embodiments, the disclosed design is appealing because it maps closely to a product that can be prototyped with modest resources. A licensor or acquirer often values an invention more highly when there is a credible engineering path from disclosure to demonstrator and from demonstrator to field pilot.

The platform may also support training-tier variants, demonstration kits, and partner evaluation units. A patent that contemplates those real-world business steps is often easier to market than one that speaks only at a theoretical level.

The detailed treatment of reader logging, calibration, and serviceability also means that the invention can fit into quality-managed organizations. This is a significant commercial point because sophisticated partners often demand traceability and maintainability rather than mere assay novelty.

In some embodiments, the cartridge may be tailored for high-throughput clinic batches, while another version is optimized for low-volume distributed use. The current specification is rich enough to support such differentiation without losing fidelity to the central point-of-care detection concept.

An important strength of the application is that it does not rely for its relevance on any single software interface, reimbursement pathway, or remote network architecture. Instead, those can remain optional layers around a concrete physical module. This can help keep the core patent more durable and more easily explainable.

In some embodiments, the platform may be paired with implementation support materials that map result categories to local follow-up guidance. That pairing can make the technology more attractive in practice without requiring the patent claims to drift away from the underlying device and assay nucleus.

Where a market package is assembled, the present application can sit alongside know-how relating to reagent formulation, calibration workflows, manufacturing tolerances, and validation planning. The value of that package is strengthened when the patent itself is technically coherent and commercially intelligible, as intended here.

The specification also contemplates that future claim sets may selectively emphasize specific commercial differentiators such as closed waste handling, compact optics, subtype-grouped output, or same-visit workflow. These are practical differentiators that competitors and partners alike are likely to care about.

In some embodiments, the reader may be field-updatable under controlled conditions so that assay refinements can reach the installed base. Such updateability is commercially useful but is described here in a way that remains subordinate to the tangible reader and cartridge architecture.

An additional source of value lies in the fact that the disclosed platform can be communicated to non-technical stakeholders. A market-facing explanation of a sealed cartridge, a compact reader, and a same-visit triage result is much easier to convey than a diffuse multi-technology platform story.

For asset-package purposes, the present filing is designed to function as a “core module patent” around which commercialization and continuation strategy can be organized. This kind of disciplined patent core can make a portfolio appear more deliberate and higher quality.

In some embodiments, the platform can be positioned as solving a workflow bottleneck rather than merely adding another laboratory assay. That positioning can matter to buyers or licensees because bottleneck-solving technologies are often easier to integrate into real programs.

In some embodiments, quality-control behavior is itself a meaningful product differentiator. A platform that declines to issue uncertain results can be more trusted than one that simply outputs categories regardless of context. The specification therefore gives quality control and invalid suppression significant prominence.

Because point-of-care molecular platforms often live or die on reproducibility, the disclosure repeatedly returns to alignment, calibration, tolerances, and controlled handling. This emphasis is intentional and is part of the reason the application can serve as a serious prosecution and market-facing asset.

In some embodiments, the patent may be licensed into a broader women's health portfolio that includes other non-overlapping innovations. The present disclosure is suitable for that role because it is narrow enough to be technically intelligible yet broad enough to support a family of practical product variants.

The cartridge-reader concept also lends itself to staged proof-building. Initial bench evidence can focus on chemistry and optics, later work can focus on cartridge manufacturing and workflow, and still later work can address deployment and quality systems. The patent is drafted to remain relevant through those stages.

In some embodiments, the platform is attractive precisely because it avoids dependence on a heavy laboratory footprint. The current application makes that theme concrete by tying the assay to compact optics, defined thermal control, and closed consumables suited to decentralized use.

Even when additional improvements are later made, the present application can remain the reference point for the core commercial story. That anchoring function can have significant value when a company wishes to explain its IP architecture to partners, investors, or acquirers.

In some embodiments, the value proposition includes recurring reagent and cartridge revenue rather than one-time device sales alone. A patent that clearly supports both the consumable and the reader sides of the platform can therefore be more valuable than one focused on only one element.

The present specification is also well suited to selective narrowing during prosecution. Because there is substantial disclosure density around the core architecture, claims can be tightened in several meaningful ways without becoming empty or commercially irrelevant.

In some embodiments, a party may commercialize an initial limited panel and later add adjacent panel options while preserving the same installed reader. This expandability can improve the long-term economic story around the platform, and the application is drafted to support it.

The platform has significance not only because cervical cancer triage is important, but because the disclosed system offers a plausible path to same-visit molecular information in formats that are compatible with real operational settings. That convergence of importance and practicality is a hallmark of a valuable patent asset.

The application also supports the preparation of structured asset materials such as claim trees, diligence summaries, product-roadmap overlays, and licensing memoranda. Such materials are easier to prepare when the base specification is this detailed and coherent.

From a portfolio-governance perspective, the present filing can help prevent future over-breadth by preserving a disciplined technical center. Later filings can branch outward intentionally rather than by accident. This makes the overall IP architecture appear more strategic and higher quality.

In some embodiments, the reader can be offered under placement or managed-service arrangements while the consumables remain the primary recurring revenue source. The patent supports these possibilities indirectly by disclosing both reader and consumable in commercially realistic detail.

The invention therefore occupies a useful strategic position: specific enough to be patentable and buildable, broad enough to support continuation and commercialization flexibility, and clear enough to be understood by sophisticated market participants. The present specification has been drafted to reinforce that position throughout.

### Additional Women's Health and Deployment Definitions

As used herein, “same-visit” means that a clinically useful result is generated in a timeframe compatible with action while the patient remains within the encounter workflow, whether physically co-located or within an actively supervised telehealth or care-coordination session.

As used herein, “point-of-care” or “POC” means near-patient deployment in a clinic, community screening site, physician office, pharmacy, mobile program, urgent-care location, or other workflow in which sample collection and result generation occur without dependence on distant central-laboratory batching.

As used herein, “triage result” includes a categorical, semi-quantitative, or workflow-oriented output usable to guide repeat sampling, follow-up, referral, counseling, escalation, or reimbursement handling.

As used herein, “high-risk HPV” includes one or more oncogenic HPV subtypes including HPV16 and HPV18 and may include pooled reporting of additional high-risk classes in non-limiting implementations.

As used herein, “quantum-dot reporter” includes fluorescent semiconductor nanocrystal labels, derivatives, or equivalent reporter constructs configured to produce a readable optical signal with a peak emission about the stated wavelength.

As used herein, “reader” includes a dedicated handheld reader, benchtop analyzer, docking device, phone-coupled optical base, or equivalent excitation-and-detection instrument configured to operate with the disclosed cartridge.

As used herein, “network deployment interface” refers to a portal, secure API endpoint, clinical dashboard, emergency coordination endpoint, or reimbursement-processing interface capable of receiving data or workflow instructions generated by the point-of-care system.

As used herein, reference to exemplary domain names such as beipoct.com, medicalcenter.us, 120.us, and medprc.com is non-limiting and is intended to illustrate concrete deployment pathways, not to restrict the invention to a particular web address.

Unless expressly stated otherwise, numerical values and ranges are approximate and are provided as practical design anchors, not as implied disclaimers of equivalent engineering variation that remains consistent with the claims.

### Network Communications and Secure Routing

In selected embodiments the reader or a communicatively coupled device transmits result data, metadata, or routing instructions to a remote endpoint.

Such transmission may be event-driven, rule-driven, user-confirmed, consent-gated, or any combination thereof.

The transmitted payload may include a result category, subtype call, internal-control status, timestamp, cartridge identifier, operator identifier, run ID, clinic ID, or referral code.

Sensitive data may be encrypted in transit and at rest using conventional cryptographic methods suitable for healthcare environments.

The system may maintain an audit record for local runs, transmitted events, user acknowledgments, and downstream workflow status changes.

In some embodiments the reader transmits only a triage category and a tokenized run reference; in other embodiments additional data are transmitted according to consent and jurisdictional policy.

Communications may be direct or may pass through a secure middleware layer that validates authentication, policy, routing rules, and endpoint availability.

These network functions do not define the narrowest technical novelty of the molecular detection core, but they contribute materially to commercial usefulness and to deployment in regulated service pathways.

In several embodiments the network layer is implemented by domain-associated service portals, which are described below as concrete non-limiting examples.

### Exemplary Domain-Specific Network Deployment Embodiments

In one exemplary deployment embodiment the point-of-care reader transmits result data, workflow status, or counseling prompts to a cloud or edge service accessible through beipoct.com.

The beipoct.com embodiment may function as a field-operator interface, patient-facing result portal, or screening-program coordination console.

In some implementations beipoct.com presents a simple triage dashboard showing result category, time to result, run validity, and next-step instructions appropriate for same-visit use.

The portal may further support patient identifiers, scheduling links, consent records, language localization, clinic-specific routing rules, or educational messaging.

In selected workflows the beipoct.com embodiment serves as the first digital layer above the molecular test and therefore acts as the practical front-end of the women's health triage pathway.

In another exemplary deployment embodiment the system routes specialist-review packages, referral summaries, or follow-up instructions to a provider-facing portal accessible through medicalcenter.us.

The medicalcenter.us embodiment may support gynecology review, colposcopy referral, pathology review, telehealth consultation, breast-health follow-up, or longitudinal care management.

In certain embodiments medicalcenter.us receives a structured payload generated from the same-visit triage engine and allows a specialist to confirm, annotate, defer, or escalate the next step.

The portal may further associate the molecular result with optional adjunct inputs such as AI-assisted cytology, breast imaging findings, or liquid-biopsy reports when those adjuncts are available.

Thus medicalcenter.us serves as a clinically meaningful extension point that broadens the value narrative of the platform without disturbing the molecular point-of-care claim core.

In another exemplary deployment embodiment urgent or high-priority triage outputs are routed to an emergency-coordination workflow associated with 120.us.

The 120.us embodiment is not limited to ambulance dispatch and may instead represent a coordination endpoint for urgent review, expedited scheduling, transport support, or acute-case escalation according to program policy.

Although the core cervical molecular workflow will often not require emergency routing, the architecture is designed so that high-priority downstream signals or integrated adjunct inputs can activate an escalation pathway when appropriate.

The ability to route certain outcomes to 120.us or a similar endpoint illustrates how the molecular node can participate in a broader care coordination network while still remaining a coherent point-of-care test system.

This deployment embodiment may be particularly useful in mobile programs, rural pathways, or cross-institution workflows that require structured escalation from local detection to higher-acuity coordination.

In another exemplary deployment embodiment pricing, coverage, and reimbursement interactions are supported through an interface associated with medprc.com.

The medprc.com embodiment may receive coded result categories, test identifiers, service metadata, route selections, or other reimbursement-support information generated by the point-of-care system.

In some implementations medprc.com provides pricing guidance, claim support logic, coverage checks, or patient-cost communication linked to the same-visit molecular result.

This integration improves commercial deployability because it connects the technical output of the cartridge-reader system to the administrative and financial realities of real-world screening programs.

The medprc.com embodiment is therefore a commercially important but non-limiting deployment layer that strengthens transaction value and operational completeness without redefining the hardware claim core.

### Optional AI-Assisted Cervical Cytology Embodiments

In optional embodiments the molecular triage node interoperates with an AI-assisted cervical cytology pipeline that receives digital cytology images and returns review information or concordance flags.

Such AI assistance may use convolutional, transformer-based, or hybrid image-processing models and may be employed as a follow-up input, a concordance check, or a triage modifier according to local policy.

The optional AI embodiment is not required to practice the presently claimed molecular system but is disclosed to preserve broader strategic and deployment value.

In one non-limiting workflow, a positive or indeterminate molecular output can trigger digital cytology review, while a negative molecular output may defer such review unless clinical factors indicate otherwise.

The system may transmit a routing token, case package, or worklist entry to an AI-assisted cytology environment while preserving the core same-visit result produced by the reader.

### Optional Breast-Health and Multi-Modal Imaging Embodiments

In optional embodiments the women's health architecture extends beyond cervical screening and interoperates with breast-health workflows including digital mammography, contrast-enhanced spectral mammography, digital breast tomosynthesis, ultrasound, MRI review, or combinations thereof.

These breast-health extensions are disclosed as optional deployment pathways rather than as the narrowest claimed inventive core of the present filing.

A provider portal such as medicalcenter.us may associate a molecular triage event with breast-health referral rules, especially where the deployment is part of a broader women's oncology program.

In one non-limiting example a high-risk cervical result or combined patient-history profile triggers an invitation for additional breast-health review or scheduling, without requiring the molecular cartridge itself to perform breast analysis.

This preserves the possibility of integrated women's oncology narratives while avoiding unnecessary diffusion of the core molecular claim set.

### Optional Liquid Biopsy and Epigenetic Embodiments

In optional embodiments the system interoperates with liquid-biopsy workflows that analyze circulating nucleic acids, circulating tumor cells, protein markers, or other blood-or fluid-derived indicators.

In optional embodiments the system also interoperates with epigenetic biomarker workflows including DNA methylation, histone-related features, or other molecular signatures that may be useful in a women's oncology pathway.

These adjunct evidence streams may be linked through the network layer, provider portal, or case-routing engine, rather than being required within the minimum cartridge implementation.

The present specification intentionally preserves such expansions as optional embodiments so that future filings may branch into narrower child applications if desired.

Optional liquid-biopsy and epigenetic extensions can increase transaction value and strategic breadth even where the presently claimed core remains focused on the HPV molecular triage node.

### INDUSTRIAL APPLICABILITY AND CLINICAL USE CONTEXTS

The disclosed system is suitable for outpatient gynecology practices, primary-care clinics, mobile women's health programs, community screening initiatives, pharmacy-based health sites, public-health campaigns, employer clinics, and low-resource settings.

The same-visit workflow is particularly useful where patient travel burden, fragmented follow-up, or delayed central-laboratory turnaround leads to avoidable loss to follow-up.

The network-enabled embodiments further support distributed care models in which a frontline collection site produces the molecular result and a separate provider site handles specialist review or downstream management.

The technology is also useful in portfolio terms because the molecular node can stand alone as a product while the network and optional deployment layers create service, licensing, and routing value.

The invention is therefore meaningful both as a practical diagnostic system and as a foundation for broader women's health deployment architectures.

### Advantages

The disclosed architecture provides a technically concrete molecular point-of-care core while preserving broad deployment and transaction value.

The cartridge-reader-kit design supports a genuine product form that can be manufactured, tested, licensed, or packaged in an asset portfolio.

The same-visit triage logic improves clinical usefulness relative to delayed-result pathways.

The network deployment embodiments connect the technical result to provider review, emergency escalation, and reimbursement systems.

The specification preserves optional pathways involving AI, breast-health workflows, liquid biopsy, and epigenetic analysis without forcing those expansions into the narrowest claim core.

This balance makes the disclosure useful for prosecution, licensing, deployment, and portfolio strategy at the same time.

### Interpretation and Non-Limiting Clauses

Unless expressly stated otherwise, the order of steps recited in examples or methods is non-limiting and steps may be combined, subdivided, omitted, or reordered where technically appropriate.

Reference to specific portals, domains, programs, institutions, or routing destinations is exemplary and non-limiting.

Reference to cervical samples includes swab-derived, eluted, or otherwise prepared cervical material compatible with the disclosed assay chemistry.

Reference to same-visit routing includes immediate on-site action and supervised near-term routing performed within the encounter workflow.

Reference to optional adjunct technologies does not imply that such technologies are required for practice of the claimed invention unless expressly recited.

The scope of protection is defined by the claims and their lawful equivalents, not by any isolated example, domain name, or implementation preference.

### Additional Engineering Supporting Embodiments

In a preferred implementation, the stated channel geometry, reaction temperature, reaction interval, and reporter-emission band are selected as a coordinated engineering window rather than as disconnected laboratory variables. The geometry, thermal profile, optical readout, and result-categorization logic are configured to cooperate in a compact cartridge-reader product intended to provide a same-visit molecular triage output that can be acted on within cervical and breast cancer care pathways.

The approximately 100-micrometer width and approximately 50-micrometer depth of at least one principal reaction microchannel provide a practical balance among reagent economy, capillary or pressure-assisted fill behavior, thermal transfer efficiency, and stable optical interrogation. A narrower channel can increase fill sensitivity or clog risk, while a materially larger channel can increase reagent burden, thermal lag, and optical variability, so the disclosed dimensions are useful as a manufacturable and performance-oriented design anchor.

Incubation of the CRISPR-Cas13a reaction at about 37 degrees Celsius for about 15 minutes is useful because it supports clinically relevant turnaround while preserving a reader architecture that is simpler, lower-power, and more field-deployable than systems requiring substantially hotter or longer-running reactions. The disclosed temperature-and-time combination therefore functions both as a biochemical operating point and as a product-design constraint that improves practical deployability.

A quantum-dot reporter with a peak emission at about 605 nanometers is particularly useful in the disclosed system because it can provide a bright, stable, and reader-compatible signal in a compact optical stack. In representative embodiments, the excitation source, filtering arrangement, detector selection, and read-window geometry are chosen to cooperate with this emission band so that the cartridge and reader operate as a coordinated optical appliance rather than as a generic fluorescence platform.

In representative embodiments, target-selection logic favors subtype-discriminating or grouped high-risk HPV motifs that preserve clinically useful stratification without forcing the instrument into an unduly broad or slow assay panel. Separate or partially separate regions for HPV16, HPV18, pooled high-risk HPV, and internal control allow the system to generate a categorical output that is more actionable than a simple positive/negative signal while remaining compatible with a compact reader and a same-visit workflow.

Internal-control function is preferably treated as a first-order safety and validity feature rather than as an optional afterthought. In useful embodiments, the processor is configured to suppress issuance of a substantive positive or negative output when the internal control does not satisfy a predetermined acceptance criterion, and instead to issue an invalid or repeat-test recommendation. This invalid-suppression behavior reduces the risk of misleading outputs in decentralized or non-laboratory deployment settings.

Threshold logic may be implemented using region-specific thresholds, background-subtracted values, normalized ratios, time-window checks, or combinations thereof. Such logic may be stored in reader memory, associated with cartridge-lot identifiers, or tied to assay-version metadata. The purpose of this logic is to make the disclosed output categories reproducible and supportable across multiple readers, multiple cartridge lots, and multiple deployment environments, thereby strengthening both enablement and commercial reliability.

Calibration may include use of cartridge identifiers, optical references, baseline checks, pre-read verification, lot-specific adjustment factors, temperature-readiness checks, or combinations thereof. In useful embodiments, the system records run identifiers, lot information, and calibration-relevant metadata together with the result category so that local or fleet-level review can distinguish assay-related issues from reader-related issues. This strengthens the practical identity of the invention as a buildable instrument-and-consumable platform.

Run-validity decision rules may distinguish among chemistry failure, fluid-transfer failure, reader-readiness failure, optical-baseline failure, and internal-control failure. In some embodiments, these determinations are made using a combination of self-check routines and reaction-region signal patterns. This allows the system to recommend repeat testing, reader service, cartridge replacement, or scheduled follow-up in a more specific manner than a single undifferentiated error state.

Clinical routing remains subordinate to the molecular core, but is commercially important because the value of a same-visit result increases when the result can be converted into a concrete next step. In representative embodiments, categorical outputs are mapped to immediate follow-up, scheduled follow-up, repeat collection, provider review, referral workflow, or reimbursement handling, while the underlying cartridge-reader-CRISPR-quantum-dot nucleus remains the primary technical anchor of the invention.

The disclosed architecture is also useful as a transferable product module because the same fundamental consumable-and-reader nucleus can support handheld, benchtop, mobile, partner-branded, or private-label implementations without changing the essential reaction geometry, thermal window, reporter band, or triage logic. This modularity increases licensability and assignment value because a transaction counterparty can integrate the disclosed module into an existing product family without first redesigning the core assay appliance.

Even where later product generations alter enclosure design, accessory workflow, communication layer, or selected assay refinements, the presently disclosed invention continues to provide a concrete nucleus defined by coordinated microfluidic geometry, controlled CRISPR-Cas13a reaction conditions, quantum-dot optical readout, internal-control-governed validity logic, and same-visit categorical triage output. This coordinated nucleus is intended to solve the practical pain point of delivering a rapid, clinically actionable molecular result in a form that can be manufactured, deployed, licensed, and incorporated into cervical and breast cancer care pathways.

1. A quantum-dot-enhanced CRISPR-Cas13a microfluidic molecular triage system for rapid genotyping of high-risk human papillomavirus (HPV) from a cervical sample, comprising: disposable microfluidic cartridge comprising a sample inlet, a lysis zone, a CRISPR reaction zone, an optical detection zone, and a waste reservoir; and a reader configured to receive the cartridge and comprising a heater, an optical excitation-and-detection assembly, and a processor; wherein the CRISPR reaction zone includes at least one reaction microchannel having a width of about 100 micrometers and a depth of about 50 micrometers; wherein the system is configured to incubate a CRISPR-Cas13a assay at about 37 degrees Celsius for about 15 minutes; and wherein the optical detection zone is configured to detect fluorescence from a quantum-dot reporter having a peak emission at about 605 nanometers and the processor is configured to generate a same-visit triage result indicating at least one of HPV16/18 positive, other high-risk HPV positive, negative, invalid, or repeat-test recommended.

2. The system of claim 1, wherein the cartridge comprises separate reaction regions corresponding to HPV16, HPV18, pooled high-risk HPV, and an internal control.

3. The system of claim 1, wherein the cartridge is single-use and sealed after sample loading.

4. The system of claim 1, wherein the cartridge comprises a polymeric substrate selected from cyclic olefin copolymer, cyclic olefin polymer, polycarbonate, and polymethyl methacrylate.

5. The system of claim 1, wherein the heater is configured to maintain the CRISPR reaction within a tolerance band around 37 degrees Celsius during the incubation.

6. The system of claim 1, wherein the processor suppresses issuance of a positive or negative result and outputs invalid or repeat-test recommended when an internal control fails a predetermined acceptance criterion.

7. The system of claim 1, wherein the optical excitation-and-detection assembly comprises at least one light source, at least one optical filter, and at least one photodetector aligned with a cartridge read window.

8. the system of claim 1, further comprising a communications interface configured to transmit the triage result or associated run metadata to a patient portal, provider portal, clinical coordination endpoint, pricing interface, reimbursement interface, or combinations thereof.

9. The system of claim 1, wherein the reader or a communicatively coupled device displays the triage result together with a same-visit routing instruction, follow-up recommendation, or workflow status indicator.

10. A method for rapid high-risk HPV genotyping and same-visit triage using a cervical sample, comprising: introducing the cervical sample into a disposable microfluidic cartridge comprising a sample inlet, a lysis zone, a CRISPR reaction zone, an optical detection zone, and a waste reservoir; lysing the cervical sample on-chip or in a chamber fluidically coupled to the cartridge; transporting lysed sample into at least one reaction microchannel having a width of about 100 micrometers and a depth of about 50 micrometers; incubating a CRISPR-Cas13a reaction in the cartridge at about 37 degrees Celsius for about 15 minutes; optically detecting fluorescence from a quantum-dot reporter having a peak emission at about 605 nanometers; and generating, with a processor, a same-visit triage result indicating at least one of HPV16/18 positive, other high-risk HPV positive, negative, invalid, or repeat-test recommended.

11. The method of claim 10, further comprising verifying an internal control before issuing the triage result.

12. The method of claim 10, wherein the optical detection comprises interrogating separate reaction regions corresponding to HPV16, HPV18, pooled high-risk HPV, and an internal control.

13. The method of claim 10, further comprising generating invalid or repeat-test recommended output when the internal control fails a predetermined acceptance criterion or when signal-to-noise criteria are not satisfied.

14. The method of claim 10, further comprising storing or exporting a run log associated with assay version, cartridge lot, and result category.

15. The method of claim 10, further comprising transmitting the triage result or associated run metadata to a remote endpoint associated with patient-facing review, provider-facing review, emergency coordination, pricing handling, reimbursement handling, or combinations thereof, and presenting a same-visit routing instruction based on the triage result.

16. A kit for quantum-dot-enhanced CRISPR-Cas13a microfluidic molecular triage of a cervical sample, comprising: a disposable microfluidic cartridge comprising a sample inlet, a lysis zone, a CRISPR reaction zone, an optical detection zone, and a waste reservoir, wherein the CRISPR reaction zone includes at least one reaction microchannel having a width of about 100 micrometers and a depth of about 50 micrometers; CRISPR-Cas13a reagents configured for incubation at about 37 degrees Celsius for about 15 minutes; at least one quantum-dot reporter having a peak emission at about 605 nanometers; and instructions for use with a reader configured to generate a same-visit triage result.

17. The kit of claim 16, further comprising an internal control reagent or control region corresponding to run-validity checking.

18. The kit of claim 16, further comprising a positive control, a negative control, cervical swabs, and lysis buffer containers.

19. The kit of claim 16, wherein the kit is packaged for same-visit cervical screening in an outpatient clinic, community screening site, mobile program, pharmacy, physician office, or combinations thereof.

20. The kit of claim 16, wherein the instructions describe transmission of triage results to a patient portal, provider portal, emergency coordination interface, pricing interface, reimbursement interface, or combinations thereof, and operation as a first-access molecular triage node within a broader clinical routing architecture.
